(19) United States
(12) Patent Application Publication
(10) Pub. No.: US 2025/0382633 A1
(43) Pub. Date: Dec. 18, 2025

(54) ADENOVIRAL VECTORS

(71) Applicant: Universität Zürich, Zürich (CH)

(72) Inventors: Patrick Christian FREITAG, Zürich (CH); Fabian BRANDL, Möhlin (CH); Dominik BRÜCHER, Zürich (CH); Fabian WEISS, Zürich (CH); Sheena Nichole SMITH, Bubendorf (CH); Birgit DREIER, Adlikon (CH); Andreas PLÜCKTHUN, Zürich (CH)

(21) Appl. No.: 18/718,620

(22) PCT Filed: Dec. 19, 2022
(86) PCT No.: PCT/EP2022/086770

§ 371 Date: Jun. 11, 2024

(30) Foreign Application Priority Data
Dec. 21, 2021 (EP) 21216348.9
Sep. 2, 2022 (EP) 22193585.1

(51) Int. Cl. C12N 15/86; C07K 14/005; C07K 16/32

(52) U.S. Cl. CPC C12N 15/86; C07K 14/005; C07K 16/32; C07K 2317/622; C12N 2710/10043; C12N 2795/00022

(57) ABSTRACT

Disclosed herein is an adenoviral vector system utilizing DARPin adapters. The system is highly effective, safe and able to deliver DNA in a cell-specific manner. It is demonstrated that the system is unexpectedly versatile, and can be used in conjunction with protein scaffolds, bioactive peptides and small molecules. This makes the system useful for numerous purposes, including the use of the system for therapeutic and diagnostic purposes.

This patent application is the U.S. National Stage of PCT/EP2022/086770 filed Dec. 19, 2022, which claims the benefit of priority from EP 22193585.1 filed Sep. 2, 2022 and EP 21216348.9 filed Dec. 21, 2021, the content of each of which is incorporated by reference herein in its entirety.

### STATEMENT REGARDING ELECTRONIC FILING OF A SEQUENCE LISTING

A Sequence Listing in XML text format, submitted under 37 C.F.R. § 1.821-1.834, entitled “VEC001PCT_Sequence listing.xml”, 12,149 bytes in size, generated May 7, 2024, and filed via Patent Center, is provided in lieu of a paper copy. This Sequence Listing is hereby incorporated by reference into the specification.

### FIELD OF THE INVENTION

Disclosed herein is an adenoviral vector system utilizing DARPin adapters. The system is highly effective, safe and able to deliver DNA in a cell-specific manner. It is demonstrated that the system is unexpectedly versatile, and can be used in conjunction with protein scaffolds, bioactive peptides and small molecules. This makes the system useful for numerous purposes, including the use of the system for therapeutic and diagnostic purposes.

### BACKGROUND

Gene therapy is a fast-growing field of biomedical research. Several thousand clinical trials are ongoing or completed. This rapid progress was possible due to achievements in DNA delivery methods, such as physical gene transfer, synthetic nanoparticles, and viral, or cellular vectors. Especially for in vivo applications, viral vectors have been shown to achieve high transduction rates and prolonged expression. Adenoviral vectors, in particular, are one of the most frequently applied gene vectors and are currently investigated for multiple clinical purposes, including but not limited to the fields of vaccines, oncology, or rare diseases. Among the more than 100 human adenovirus serotypes, the most prominent and studied adenovirus serotype is the human adenovirus serotype C5 (HAdV-C5).

Multiple generations of HAdV-C5 vectors have been developed, including non-replicative ones such as first-generation or helper-dependent adenoviral vectors, as well as replicative adenoviral vectors which all differ in their genomic content. However, the protein content, especially the outer capsid of all HAdV-C5-derived vectors dictates the tropism of the vector. Unmodified, the HAdV-C5 enters the cell by binding the coxsackievirus and adenovirus receptor (CAR), followed by the interaction of the RGD motif of the adenovirus penton base with αVβ3/β5 integrins on the cell surface. Additionally, the virus can be taken up by cells though interactions with scavenger receptor and heparan sulfate proteoglycans. Although this natural uptake process has been reported to lead to success for specific cell types, unmodified vectors are limited by their specificity for only a few cell surface receptors, restricting adenoviral gene delivery to cell and tissue populations that express the receptors governing the natural tropism.

To overcome these limitations, various chimeras or capsid-engineering methods have been applied (J. Virol. 71, 4782-4790 (1997); Exp Hematol 32, 536-546 (2004); J. Virol. 82, 630-7 (2008); J. Virol. 70, 6839-6846 (1996)). However, the manipulation of the protein structure and the incorporation of additional binding proteins encoded on the viral genome is not only a tedious process, but can drastically reduce viral titers and possibly lead to unpredictable interferences, ultimately resulting in additional validation and purification issues for each target to be investigated (Mol. Ther. 12, 107-117 (2005); J. Gene Med. 4, 356-370 (2002); FEBS Lett. 594, 1918-1946 (2020)).

The cumbersome optimization resulting from genomic alteration of viral DNA led to developments of different bispecific proteins binding HAdV-C5 capsid proteins and a cellular receptor of choice. These exogenously produced proteins are manipulating the HAdV-C5 interactions, while leaving the viral genome unmodified (Hum. Gene Ther. 21, 739-749 (2010); Hum. Gene Ther. 23, 70-82 (2012); Adv. Cancer Res. 115, 39-67 (2012)). To be successfully applied, retargeting bispecific proteins require a very strong binding interaction with the vector, preventing dissociation from the viral capsid which would result in untargeted virions. Additionally, off-target transduction should be inhibited by blocking natural binding tropism, ensuring only specific vector uptake.

Binding the knob domain of the HAdV-C5 fiber protein offers the possibility to fulfill these criteria, since a major HAdV-C5 attachment interaction is mediated through the knob domain of the HAdV-C5 fiber protein. Fiber-binding bispecific proteins have the additional advantage that they are released after endosomal escape, and thus do not interfere with intracellular viral trafficking (Annu. Rev. Virol. 6, 177-197 (2019)).

HAdV-C5-binding bispecific proteins which form a stable trimeric adapter and at the same time inhibit coxsackievirus and adenovirus receptor tropism by binding the knob as a clamp without any detectable off-rate have been described (Proc. Natl. Acad. Sci. 110, E869-E877 (2013). These adapters consist of two different designed ankyrin repeat proteins (DARPins) and a trimerization module (Annu. Rev. Pharmacol. Toxicol. 55, 489-511 (2015)). The retargeting module specifically binds to a cell-surface marker and is fused to the knob-binding module (DARPin 1D3nc) via a (G₄S)₄ linker. A crystal structure of 1D3nc in complex with the fiber knob demonstrated direct interference of the DARPin with CAR-binding to the fiber knob. Furthermore, the knob-binding entity is connected to the trimerization module, formed by the protein SHP from lambdoid phage 21. This results in adapter binding to the HAdV-C5 knob as a chelate.

Aforementioned adapters, however, still suffer from many limitations. First, for many targets no DARPins have been identified as of today, thereby limiting the number of possible targets or requiring elaborate new selection campaigns. Second, genetic fusions to the adapter limit possible retargeting modules to encodable fusions, i.e., proteins or peptides, even though a multitude of medically relevant interaction partners are synthetic small molecules that have to be obtained by chemical synthesis. Third, only single binding entities have been reported.

Certain-DARPin-based fusion protein have been described in the scientific literature. For example US2015/0232573 describes anti HER2 antibodies fused to anti-EGFR DARPins. Such approaches relate to isolated molecules. They are expressed in bacteria. In the current system the adapters are used in a multilayer context. Complex trimeric structures are expressed and formed in mammalian cells. Despite such trimeric structure, the present disclosure provides a possibility to express functional adapter molecules in mammalian cells and then be used as a multimer (trimer) for the redirection of adenoviruses, rather than monomer and then used as a direct single agent.

The present invention solves these problems by providing novel adapter molecules that overcome aforementioned shortcomings. Despite the fact that the adapters disclosed in the prior art are known for nearly a decade, no attempts have been to extend or replace the retargeting domain of the known adapters with moieties other than DARPins and for binding to targets other than overexpressed surface receptors. Contrary to the belief in the community it is shown that the adapters also function with antibody fragments, bioactive peptides or small molecules genetically fused or chemically coupled to or replacing the retargeting domain, thereby providing completely new avenues for further therapeutic intervention using adenoviral vectors.

### SUMMARY OF THE INVENTION

The present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers,
with the proviso that the protein scaffold is not a designed ankyrin repeat domain. Preferably, when the second designed ankyrin repeat domain of the second module is not present, then the protein scaffold is not a designed ankyrin repeat domain.

Said first module and said second module may be separated by a flexible linker. Said second module may be N-terminal of said first module.

In certain embodiments the recombinant protein of the present disclosure comprises from the N-to the C-terminus
a) a designed ankyrin repeat domain which binds to the knob of an adenovirus,
b) a short linker,
c) trimerization domain,
d) a flexible linker,
e) optionally a second designed ankyrin repeat domain, and
f) a protein scaffold, a bioactive peptide or a small organic molecule.

Said designed ankyrin repeat domain of said first module binds to the knob of adenovirus serotype 5. Said trimerization domain of said first module may or may be derived from the capsid protein SHP of lambdoid phage 21. Said protein scaffold is an antibody fragment. Said antibody fragment is a scFv.

In certain embodiments, said second designed ankyrin repeat domain of said second moldule is present. In certain embodiments, said second designed ankyrin repeat domain of said second moldule is absent.

The present disclosure also relates to nucleic acids encoding aforementioned recombinant proteins. The present disclosure also relates to vectors comprising aforementioned nucleic acids. The present disclosure also relates to an adenovirus comprising aforementioned recombinant proteins, nucleic acids or vectors. The present disclosure also relates to an eukaryotic cell expressing or producing aforementioned recombinant proteins or comprising aforementioned nucleic acids, vectors, or adenoviruses. The present disclosure also relates to the use of aforementioned recombinant proteins, nucleic acids, vectors or adenoviruses in an adenoviral delivery system. The present disclosure also relates to aforementioned recombinant proteins, nucleic acids, vectors, adenoviruses or eukaryotic cells for use in medicine.

### FIGURE LEGENDS

FIG. 1 shows an SDS-PAGE demonstrating the purity of the adapter molecules. 2 μg of the respective protein fractions were heated at 92° with 0.1 M DIT prior to loading on to the gel. The lanes of the gel are labelled with the respective protein loads: 4D5 LH (scFv 4D5 in the V_{H}-linker-V_{L} orientation), DARPin G3, DARPin E2_5, G3-NT (DARPin G3 coupled to neurotensin), E2_5-NT (DARPin coupled E2_5 to neurotensin), G3-FTP3 (DARPin G3 coupled to folate), E2_5-FTP3 (DARPin E2_5 coupled to folate). The molecular weight marker is shown in the very right lane.

FIG. 2 shows a schematic overview of the different types of the trimeric adapters tested in the present invention. On the left trimeric adapter bound to the knob of an adenovirus is shown. The trimeric adapters consist of three domains: the trimeric SHP protein, the knob-binding DARPin and an exchangeable retargeting domain. Various types of exchangeable retargeting domains are shown on the right. From top to bottom: a scFv, a classical retargeting domain consisting of a retargeting DARPin, a bioactive peptide and a small molecule.

FIG. 3 demonstrates the expression of the receptors on the cell surface of target cells by flow cytometry. Expression of folate receptor alpha on KB cells was confirmed using the labeled folate derivative FolateRSense™ 680. NTR1 expression in the CHO Flp-In NTR+ cells was confirmed after tetracycline induction with fluorescently labeled peptide HL488-NTS8-13 (Sci. Adv. 7, 1-16 (2021)). The expression of HER2 on SKBR3 cells was detected using the Alexa-488 labeled antibody FAB1129G. All staining agents were tested on non-expressing CHO parental cells (light grey) and compared with the autofluorescence of the cells (medium grey) and the stained cells (dark grey).

FIG. 4 shows the transduction efficiency and specificity measured on folate receptor-expressing KB cells, HER2-expressing SKBR3 cells, and NTR-expressing CHO Flp-In cells. HAdV-C5 was coated with a knob-to-adapter ratio of 1:20 and then added to 22,500 cells with a MOI of 2.5 PFU/cell. Gene delivery was analyzed 48 h post mixture with HAdV-C5 encoding iRFP670 (upper row) or luciferase (lower row). Three separate cell populations were tested; error bars represent standard deviation.

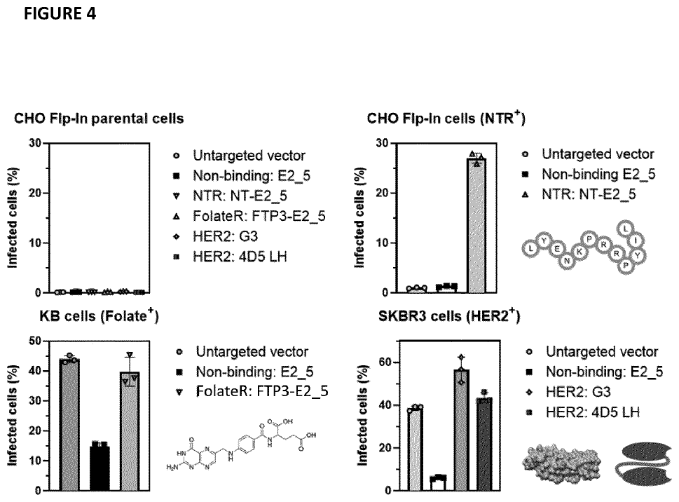

FIG. 5 shows a graphical depiction of bispecific targeting, using the G3 DARPin fused to either folate (FTP3-G3) or NT (NT-G3) as an example.

FIG. 6 shows the transduction efficiency and specificity measured on folate receptor-expressing KB cells, HER2-expressing SKBR3 cells, and NTR-expressing CHO Flp-In cells with virus retargeted to folate receptor, HER2, and NTR, respectively. HAdV-C5 was coated with a knob-to-adapter ratio of 1:20 and then added to 22,500 cells with a MOI of 2.5 PFU/cell. Gene delivery was analyzed 48 h post mixture with HAdV-C5 encoding iRFP670 (upper row) or luciferase (lower row). Three separate cell populations were tested; error bars represent standard deviation.

FIG. 7 shows GFP expression, shown as % of GFP positive cells, of RKO cells that are unmodified (naked, no adapter), or that are treated with a blocking adapter or an EGFR retargeting adapter (tumor targeted). Tumor targeting significantly increased transgene expression upon treatment of a replication-competent adenovirus 5.

FIG. 8 shows an analysis of the same data shown in FIG. 7. Shown here is the mean fluorescence.

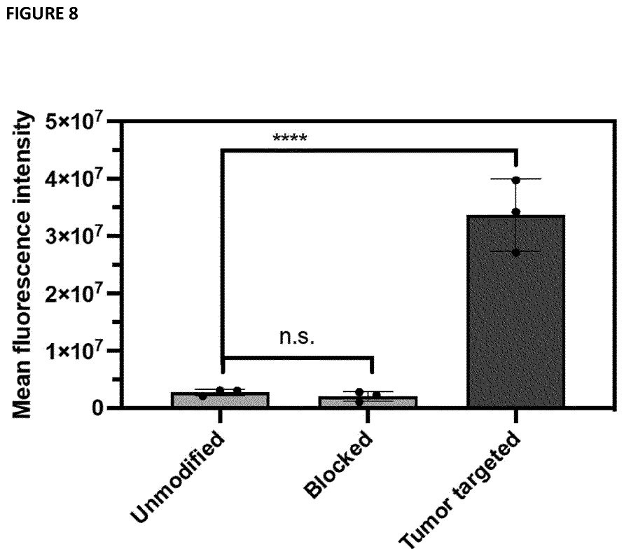

FIG. 9 shows that a second designed ankyrin repeat domain in the adapter molecules, which serves as a rigid linker, facilitates the functional expression the luciferase reporter moiety. Simple replacement of the retargeting domain with a small molecule is not sufficient for the most effective transduction of the target cell. The geometry of the DARPin serving as rigid spacer allows optimal interaction of the target receptor with the adenoviral vector, leading to effective uptake rather than just binding.

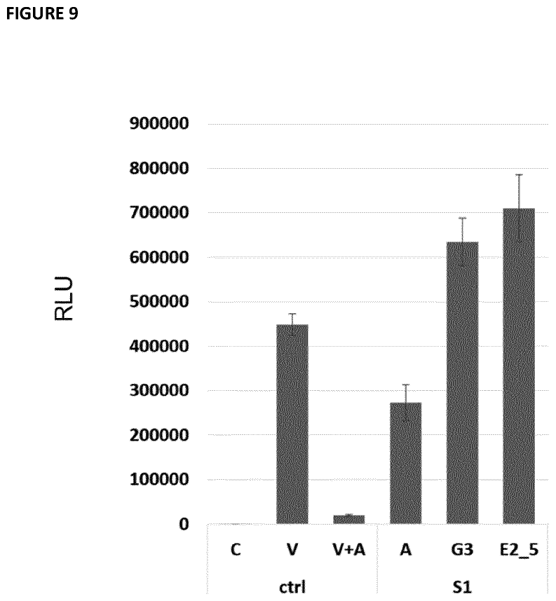

### DEFINITIONS

The term “recombinant” as used in recombinant protein, recombinant protein domain and the like, means that said polypeptides are produced by the use of recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen). When such a constructed recombinant expression plasmid is inserted into a host cell (e.g. E. coli), this host cell can produce the polypeptide encoded by this recombinant DNA. The correspondingly produced polypeptide is called a recombinant polypeptide.

The term “protein” as used herein refers to a polypeptide, wherein at least part of the polypeptide has, or is able to, acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides.

A part of a protein, which individually has, or is able to acquire a defined three-dimensional arrangement by forming secondary or tertiary structures, is termed “protein domain” or “domain”. Such protein domains are well known to the practitioner skilled in the art.

The term “polypeptide” as used herein refers to a molecule consisting of one or more chains of multiple, i.e. two or more, amino acids linked via peptide bonds. A polypeptide typically consists of more than twenty amino acids linked via peptide bonds.

The term “peptide” as used herein refers to as used herein refers to a molecule consisting of one or more chains of multiple, i.e. two or more, amino acids linked via peptide bonds. A peptide typically consists of not more than twenty amino acids linked via peptide bonds. An exemplary peptide of the present disclosure is neurotensin, a neuropeptide consisting of 13 amino acids.

The term “bioactive peptide” as used herein refers a peptide that mediate the action of sequences, molecules, or supramolecular complexes associated therewith via binding to a target molecule, thereby exerting and/or triggering an effect or a response in a target cell. Purification tags that are fused to polypeptides or proteins merely for the purpose of purifying the respective polypeptide or protein are not bioactive peptides according to the present disclosure. Neurotensin, an exemplary peptide experimentally used in the present disclosure is a bioactive peptide. Neurotensin binds to NTSR1s and is involved in the regulation of luteinizing hormone and prolactin release.

The term “purification tag” refers to short peptides that are fused to polypeptides or protein for the purpose of purifying said polypeptide or protein. The purification tag specifically binds to another moiety with affinity for the purification tag. Such moieties which specifically bind to a purification tag are usually attached to a matrix or a resin, such as agarose beads. Moieties which specifically bind to purification tags include antibodies, nickel or cobalt ions or resins, biotin, amylose, maltose, and cyclodextrin. Exemplary purification tags include histidine tags (such as a hexahistidine peptide or a MRGS(H)₆ tag), which will bind to metal ions such as nickel or cobalt ions. Other exemplary purification tags are the myc tag, the Strep tag, the Flag tag and the V5 tag.

The terms “designed ankyrin repeat protein”, “designed ankyrin repeat domain” and “DARPin” as used herein refer artificial polypeptides, comprising several ankyrin repeat motifs. These ankyrin repeat motifs provide a rigid interface arising from typically three repeated β-turns. DARPins usually carry two three repeats corresponding to an artificial consensus sequence, wherein six positions per repeat are randomized, flanked by two capping repeats with a hydrophilic surface (Gebauer and Skerra, 2009; WO 02/20565).

The term “protein scaffold” means a protein with exposed surface areas in which amino acid insertions, substitutions or deletions are highly tolerable. Examples of protein scaffolds that can be used to generate binding domains of the present invention are antibodies or fragments thereof such as single-chain Fv or Fab fragments, T cell receptor such as single chain T cell receptors, protein A from Staphylococcus aureus, the bilin binding protein from Pieris brassicae or other lipocalins, ankyrin repeat proteins, monobodies, human fibronectin, or antibodies from camelids, such as nanobodies. Protein scaffolds are known to the person skilled in the art (Curr Opin Biotechnol 22:849-57 (2011); Ann Rev Pharmacol Toxicol 60:391-415 (2020)). In certain embodiments of the present disclosure the protein scaffold is a polypeptide. In certain embodiments of the present disclosure the protein scaffold is a monomeric polypeptide. In certain embodiments of the present disclosure, the protein scaffold is an antibody fragment. In certain embodiments of the present disclosure the protein scaffold is a scFv. In certain embodiments of the present disclosure the protein scaffold is an a single chain T cell receptor. In certain embodiments of the present disclosure the protein scaffold is a peptide. In certain embodiments of the present disclosure the protein scaffold is not a designed ankyrin repeat domain. In certain embodiments of the present disclosure the protein scaffold is not a designed ankyrin repeat domain if the second module does not comprise a second designed ankyrin repeat domain.

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain variable fragment”, “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1 126-1 136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The term “single chain TCR”, “single chain T cell receptor” and “scTCR” as used herein refers to any construct containing the variable domains of a T cell receptor in single chain format. Such single chain formats include, but are not limited to the constructs described in (J Immunol Methods (1998) 221:59-76, Cancer Immunol Immunother (2002) 51:565-73, WO 2019/219709 and WO 2004/033685. In certain embodiments single chain TCR variable domains and/or single chain TCRs may have a disulfide bonds as described in WO 2004/033685.

The term “immunoglobulin” as used herein refers to any polypeptide or fragment thereof from the class of polypeptides known to the skilled person under this designation and comprising at least one antigen binding site. Preferably, the immunoglobulin is a soluble immunoglobulin from any of the classes IgA, IgD, IgE, IgG, or IgM, or a fragment comprising at least one antigen binding site derived thereof. Also comprised as immunoglobulins of the present invention are a bispecific immunoglobulin, a synthetic immunoglobulin, an immunoglobulin fragment, such as Fab, Fv or scFv fragments etc., a single chain immunoglobulin, and a nanobody. Further included are chemically modified derivatives of any of the aforesaid, e.g. PEGylated derivatives, as well as fusion proteins comprising any of the aforesaid immunoglobulins and fragments thereof. The immunoglobulin may be a human or humanized immunoglobulin, a primatized, or a chimerized immunoglobulin or a fragment thereof as specified above. Preferably, the immunoglobulin of the present invention is a polyclonal or a monoclonal immunoglobulin, more preferably a monoclonal immunoglobulin or a fragment thereof as specified above.

The terms “binds”, “is specific” and “specifically binds” as used herein refers to a molecule, for example an antibody or an antibody fragment, which recognizes a specific antigen, but does not substantially recognize or bind other molecules in a sample. An antibody that specifically binds to an antigen from one species may also bind to that antigen from one or more further species. Such cross-species reactivity does not itself alter the classification of an antibody as specific.

The term “small organic molecule” as used herein refers to a molecule of size comparable to those organic molecules generally used in pharmaceuticals. The term excludes biological macromolecules (e.g.; proteins, nucleic acids, etc.); preferred small organic molecules range in size up to 2000 Da, and most preferably up to about 1000 Da.

The term “nucleic acid” as used herein refers to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “vector” as used herein means a construct, which is capable of delivering, and usually expressing or regulating expression of, one or more gene(s) or nucleic acid(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “linker” as used herein refers a molecule or macromolecule serving to connect different moieties, modules or domains of a peptide or a polypeptide or, a protein/polypeptide domain and a non-protein/non-polypeptide moiety. Linkers can be of different nature. Different domains or modules within proteins can be linked via peptide linkers. Linkers can also be generated chemically, for example to link small organic molecules or peptides to a protein. Maleimide conjugation as used in the present disclosure is one such example.

The term “flexible linker” as used herein refers to a peptide linker linking two different domains or modules of a protein and providing a certain degree of flexibility. Preferably, the flexible linker is hydrophilic and does not interacting with other surfaces. Commonly used flexible linkers are glycine-serine linkers (Biochemistry 56(50):6565-6574 (2017)). Glycine and serine are flexible and the adjacent protein domains are free to move relative to one another. Such flexible linkers are referred to herein as “glycine-serine linkers”. Other amino acids commonly used in respective linkers are proline, asparagine and threonine. Often the linker contains several repeats of a sequence of amino acids. A flexible linker used in the present disclosure is a (Gly₄Ser)₄-linker, i.e. a linker containing four repeats of the sequence glycine-glycine-glycine-glycine-serine. Other linkers that could be used in accordance with the present disclosure include but are not limited to PAS linkers, i.e. linkers containing repeats of the sequence proline-alanine-serine (Protein Eng Des Sel (2013) 26, 489-501 and charged linkers.

The term “short linker” as used herein refers to a peptide linker linking two different domains or modules of a protein and which is no longer than four, preferably no longer than three amino acids long. More preferably the short linker is no longer than two amino acids long. Alternatively the short linker is only one amino acid long. Alternatively the short linker is a single glycine residue.

The term “chemical conjugation” as used herein refers to technologies, by which small organic molecules can be linked to polypeptides. Various technological approaches are known in the field, see for example Bioconjugate Chemistry, 26(11):2176-2185. Maleimide conjugation is one example of chemical conjugation.

The term “maleimide conjugation” as used herein refers to a method to link two different molecules with a maleimide group and a thiol group. Maleimide-thiol conjugation chemistry is a common tool in particular in bioconjugation due to its synthetic accessibility, reactivity, and practicability (Chemistry Europe 25(1):43-59 (2019)).

The term “amino acid mutation” refers to amino acid substitutions, deletions, insertions, and modifications, as well as combinations thereof. Amino acid sequence deletions and insertions include N- and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful.

The term “variant” as used herein refers to a polypeptide that differs from a reference polypeptide by one or more amino acid mutation or modifications.

The term “host cell” as used herein refers to any kind of cellular system which can be engineered to generate molecules according to the present disclosure. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Host cells according to the present disclosure can be a “eukaryotic host cell” and include yeast and mammalian cells, including murine cells and from other rodents, preferably vertebrate cells such as those from a mouse, rat, monkey or human cell line, for example HKB11 cells, PERC.6 cells, HEL293T cells, CHO cells or any type of HEK cells, such as HEK293 cells or HEK 993 cells. Also suspension cell lines like CHO-S or HEK993 cells, or insect cell cultures like Sf9 cells may be used.

Host cells according to the present disclosure can also be “procaryotic cell” and include bacterial cells, such Escherichia coli. Certain strains of Escherichia coli may be particularly useful for expression of the molecules of the present disclosure, such as Escherichia coli strain DH5 (available from Bethesda Research Laboratories, Inc., Bethesda, Md/US).

The term “module” as used herein refers to a part of a protein comprising one or more domains, linkers and/or amino acid sequence stretches.

The molecules of the present disclosure form trimers that are highly stable. Each monomer contains a domain responsible for the formation of trimers which is referred to herein as “trimerization domain”. A preferred trimerization domain is the capsid protein SHP of lambdoid phage 21 (J Mol Biol; 344(1):179-93; PNAS 110(10):E869-77 (2013)). SHP of lambdoid phage 21 has the following amino acid sequence:

(SEQ ID No. 1)
VRIFAGNDPAHTATGSSGISSPTPALTPLMLDEATGKLVVWDGQKAGSAV
GILVLPLEGTETALTYYKSGTFATEAIHWPESVDEHKKANAFAGSALSHA
ALP

The term “stable trimer” as used herein refers to a protein trimer by protein monomers comprising a trimerization domain, and wherein said trimer exhibits a stability which is higher than other, conventional protein trimers. For example, a stable trimer has a higher functional stability, a higher kinetic stability, or a higher high life for unfolding than other protein trimers. An example of a stable trimer is a trimer formed by monomers comprising the trimerization domain of the capsid protein SHP of lambdoid phage 21.

The term “derived from” in the context of an amino acid sequence refers to an amino acid sequence that is different to an original amino acid sequence, but maintains the function or activity of the original amino acid sequence.

The term “adenovirus” as used herein refers to any adenovirus, i.e. to human and non-human serotypes. The human isolates are classified into subgroups A-G. A preferred adenovirus of the present disclosure is adenovirus subtype 5 (“HAdV-C5”). HAdV-C5 includes modified version of the virus, such as replication-deficient HAdV-C5 version, e.g. containing an E1/E3 deletion and/or one or more of the 4 mutations in the HVR7 (I421G, T423N, E424S and L426Y) (Nat. Commun. 9, 450 (2018)). The adenovirus of subtype 5 may also be a virus which is capable of replicating in the host cell.

The terms “CAR” and “CXADR” as used herein refers to coxsackievirus and adenovirus receptor (UniProt: P78310). CAR is a type I membrane receptor for coxsackie viruses and adenoviruses.

The term “knob” as used herein refers to a knob on the end of the adenovirus fiber (e.g. GenBank: AAP31231.1) that binds to the cellular receptor. The knob of adenovirus subtype 5 binds to CAR. Adenoviruses having a four-amino acid deletion within the FG loop of the knob (Δ TAYT) show a decreased ability of the mutated knob to bind to CAR (Science, 286:1568-1571 (1999); J Mol Biol 405(2):410-426). This deletion is referred to herein as “TAYT mutation”.

The molecules of the present invention contain a designed ankyrin repeat domain in the first module that binds to the knob of an adenovirus. A preferred designed ankyrin repeat domain that binds to a knob is DARPin 1D3. Another preferred designed ankyrin repeat domain that binds to a knob is DARPin 1D3nc, a derivative of 1D3nc containing a stabilized C-cap. DARPin 1D3 has the following amino acid sequence:

(SEQ ID No. 2)
RSDLGKKLLEAARAGQDDEVRILMANGADANAYDHYGRTPLHMAAAVGHL
EIVEVLLRNGADVNAVDTNGTTPLHLAASLGHLEIVEVLLKYGADVNAKD
ATGITPLYLAAYWGHLEIVEVLLKHGADVNAQDKFGKTPEDLAIDNGNED
IAEVLOG

The terms “human epidermal growth factor receptor 2” and “HER2” refers a human protein encoded by the ERBB2 gene (UniProt: P04626). It is also known as CD340, HER2/neu, MLN19, NGL or TKR1. HER2 has the following amino acid sequence:

(SEQ ID No. 3)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHENHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

HER2 was used as target antigen to exemplify adapters comprising an antibody fragment, i.e. a scFv, as retargeting domain. The specific anti-HER2 scFv used is derived from humanized mouse antibody hu4D5 which carries the binding domain of the therapeutic antibody trastuzumab (Endocr. Relat. Cancer 9, 75-85 (2002)). This scfv, 4D5 LH, has the following sequence:

(SEQ ID No. 4)
DIVMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEL

The terms “neurotensin receptor 1”, “NTR1”, and “NTSR1” refers a human protein encoding a G protein-coupled receptor (UniProt: P30989). It is also known as NTRH or NTRR. NTR1 has the following amino acid sequence:

(SEQ ID No. 5)
MRLNSSAPGTPGTPAADPFQRAQAGLEEALLAPGFGNASGNASERVLAAP
SSELDVNTDIYSKVLVTAVYLALFVVGTVGNTVTAFTLARKKSLQSLQST
VHYHLGSLALSDLLTLLLAMPVELYNFIWVHHPWAFGDAGCRGYYFLRDA
CTYATALNVASLSVERYLAICHPFKAKTLMSRSRTKKFISAIWLASALLA
VPMLFTMGEQNRSADGQHAGGLVCTPTIHTATVKVVIQVNTFMSFIFPMV
VISVLNTIIANKLTVMVRQAAEQGQVCTVGGEHSTFSMAIEPGRVQALRH
GVRVLRAVVIAFVVCWLPYHVRRLMFCYISDEQWTPFLYDFYHYFYMVTN
ALFYVSSTINPILYNLVSANFRHIFLATLACLCPVWRRRRKRPAFSRKAD
SVSSNHILSSNATRETLY

NTR1 was used as target antigen to exemplify adapters comprising a bioactive peptide, neurotensin NT(8-13), as retargeting domain. NT(8-13) has the following sequence:

(SEQ ID No. 6)
RRPYIL

The terms “folate receptor a”, “FOLR1” and “FTR” refers a human protein encoding GPI-anchored protein (UniProt: P15328). It is also known as FBP or NCFTD. Folate receptor a has the following amino acid sequence:

(SEQ ID No. 7)
MAQRMTTQLLLLLVWVAVVGEAQTRIAWARTELLNVCMNAKHHKEKPGPE
DKLHEQCRPWRKNACCSTNTSQEAHKDVSYLYRFNWNHCGEMAPACKRHF
IQDTCLYECSPNLGPWIQQVDQSWRKERVLNVPLCKEDCEQWWEDCRTSY
TCKSNWHKGWNWTSGFNKCAVGAACQPFHFYFPTPTVLCNEIWTHSYKVS
NYSRGSGRCIQMWFDPAQGNPNEEVARFYAAAMSGAGPWAAWPFLLSLAL
MLLWLLS

FTR was used as target antigen to exemplify adapters comprising a small molecule, folic acid, as retargeting domain. Folic acid has the following chemical structure (CAS registry number: 59-30-3):

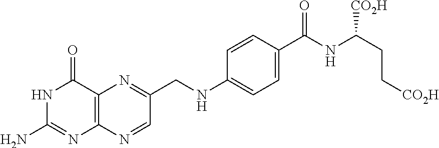 [SMILES: Nc1nc2ncc(CNc3ccc(C(=O)N[C@@H](CCC(=O)O)C(=O)O)cc3)nc2c(=O)[nH]1]

### EMBODIMENTS OF THE INVENTION

Disclosed herein is an easy-to-use and widely applicable adapter system which expands uses of adenoviral gene delivery systems available today. Although the capacity of adenoviral delivery systems has increased by the use of helper-dependent high-capacity systems, the respective technologies still have many shortcomings. One of the key advantages of the system is its versatility, overcoming the natural. restriction to targets expressing CAR or other HAdV-binding proteins. The technology also incorporates various forms of binding molecules, can use various expression systems, and allows the generation of multi-specific constructs. At the same time, it is easy to use, and due to the modular design very flexible. The system was successfully tested with various, structurally distinct adapters expressing bioactive peptides, antibody fragments and small molecules as targeting agents.

Systems available so far utilize vectors consisting of two designed ankyrin repeat domains (Proc. Natl. Acad. Sci. 110, E869-E877 (2013)). The first designed ankyrin repeat domain binds to the knob of an adenovirus, whereas the second designed ankyrin repeat domain serves as a retargeting moiety. This system has been known for almost a decade. DARPins are stable and rigid proteins that are typically resistant to misfolding and aggregation. There was hence no incentive to test whether the known adapter molecules could be modified as shown herein, since such attempts would have been considered as unsuccessful.

Herein it is demonstrated that the known adenoviral vectors can be modified in various ways. First, it is demonstrated herein that the retargeting designed ankyrin repeat domain of the constructs described in Proc. Natl. Acad. Sci. 110, E869-E877 (2013) can be replaced with moieties other than designed ankyrin repeat domains. Second, it is also demonstrated herein that the second designed ankyrin repeat domain of the constructs described in Proc. Natl. Acad. Sci. 110, E869-E877 (2013) can be extended with additional moieties other than designed ankyrin repeat domains, thereby generating constructs with novel retargeting domains. Third, it is also demonstrated herein that constructs described in Proc. Natl. Acad. Sci. 110, E869-E877 (2013) are also compatible with structurally different retargeting domains, such as antibodies, antibody fragments, small organic molecules or bioactive peptides.

In summary, contrary to the belief in the community it is shown that the adapters also function with antibodies, antibody fragments, bioactive peptides or small molecules genetically fused or chemically coupled to or replacing the retargeting domain, thereby providing completely new avenues for further therapeutic intervention using adenoviral vectors with a large capacity.

The present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

The present disclosure also relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers,
with the proviso that the protein scaffold is not a designed ankyrin repeat domain.

The present disclosure also relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

In certain embodiments, the present disclosure relates to recombinant proteins or uses of such recombinant proteins that comprise a trimerization domain. The trimerization domain is responsible for the formation of trimers. Each monomer of the molecules of the present disclosure comprises a trimerization domain. Principally any trimerization domain may be used, provided it is stable and geometrically fits the knob of the adenovirus used.

A preferred trimerization domain is the capsid protein SHP of lambdoid phage 21 (J Mol Biol; 344(1):179-93; PNAS 110(10): E869-77 (2013)).

In certain embodiments, the present disclosure relates to recombinant proteins comprising
a) a first module comprising a designed ankyrin repeat domain and a trimerization domain
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is the capsid protein SHP of lambdoid phage 21.

In other embodiments, the present disclosure relates to recombinant proteins comprising
a) a first module comprising a designed ankyrin repeat domain and a trimerization domain
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is derived from the capsid protein SHP of lambdoid phage 21.

In other embodiments, the present disclosure relates to recombinant proteins comprising
a) a first module comprising a designed ankyrin repeat domain and a trimerization domain
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain comprises the amino acid sequence of SEQ ID No. 1.

Also other trimerization domains known to the skilled person may be used for the formation or trimers. Without being limited other potential trimerization domains include the trimerization domain involved in collagen folding (Int J Biochem Cell Biol 44:21-32 (2012)), the trimerization domain of T4 phage fibritin (PLOS One 7: e43603 (2012) or the GCN4-based isoleucine zipper (J Biol Chem 290:7436-42 (2015)).

The trimerization domain is responsible for the formation of the trimeric adapter molecules. The trimers disclosed herein are extraordinary stable (J Mol Biol (2004) 344:179-93; PNAS (2013) 110 E869-77). In certain embodiments the trimeric adapter molecules of the present disclosure remain intact in SDS gel electrophoresis. In other embodiments the trimeric adapter molecules are not denatured in SDS gel electrophoresis. In other embodiments the trimeric adapter molecules have a half-life in solution of at least one week, preferably at least two week and even more preferably at least one month.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain has a half-life in solution of at least one week, preferably at least two week and even more preferably at least one month.

In certain embodiments, the present disclosure relates to recombinant proteins comprising a designed ankyrin repeat domain that binds to the knob of an adenovirus. The present disclosure can however also be practiced with other viruses. If another virus is used a designed ankyrin repeat domain needs to be selected that binds to the knob of such virus. Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of a virus, and
wherein said trimerization domain is capable of forming stable trimers.

A preferred virus to be used in the context of the present disclosure is adenovirus of serotype 5. Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus of serotype 5, and
wherein said trimerization domain is capable of forming stable trimers.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus of serotype 5, and
wherein said trimerization domain is or is derived from the capsid protein SHP of lambdoid phage 21.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus of serotype 5, and
wherein said trimerization domain comprises the amino acid sequence of SEQ ID No. 1.

In certain embodiments of the present disclosure, said adenovirus is a replication-deficient virus. In other embodiments of the present disclosure, said adenovirus is capable of replicating in the host cell. In other embodiments of the present disclosure, said adenovirus is a lytic virus. In other embodiments of the present disclosure, said adenovirus is an oncolytic virus. In certain embodiments, any of aforementioned adenoviruses is an adenovirus of serotype 5.

It will be understood that also other adenoviral serotypes may be used in the spirit of the present disclosure, including human adenovirus serotype c5 (HAdV-C5), HAd2, HAd3, HAdV-B35, HAdV-D26, as well as hybrids thereof. A list of adenoviruses can be found on the website of the Human Adenovirus Working group (http://hadvwg.gmu.edu). Also non-human adenoviruses may be used within the scope of the present disclosure, such as the AstraZeneca vaccine chimpanzee adenovirus Y25 (CHAdY25), or non-human adenoviral vectors were developed from bovine (BAd), canine (CAd), chimpanzee (ChAd), ovine (OAd), porcine (PAd), or fowl (FAd).

The recombinant proteins of the present disclosure comprise a designed ankyrin repeat domain which binds to the knob of a virus or adenovirus. It will be appreciated that any designed ankyrin repeat domain with specificity for the knob of a virus or adenovirus may be used within the spirit of the present disclosure. Exemplified herein is a designed ankyrin repeat domain derived from DARPin 1D3 (Proc. Natl. Acad. Sci. 110, E869-E877 (2013)). DARPin 1D3 binds to the knob of an adenovirus and comprises the amino acid sequence of SEQ ID No. 2. Used herein is 1D3nc, a derivative of 1D3 containing a stabilized C-cap.

Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module is or is derived from DARPin 1D3, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module comprises the amino acid sequence of SEQ ID No. 2, and
wherein said trimerization domain is capable of forming stable trimers.

It will also be understood that also variants of DAPRin 1D3 may used within the spirit of the present disclosure. In other words, the amino acid sequence of such modified DARPin 1D3 does not need to be identical to that of amino acid sequence of SEQ ID No. 2, but may contain amino acids mutations, provided that the function of DAPRin 1D3, i.e. binding to the knob of an adenovirus is preserved. Also DARPin's different than 1D3, but having the same target specificity, may be used within the scope of the present disclosure. Such new DARPin may for example be selected in a new screening campaign. Also binding entities different than DARPin's, i.e. binders based on a different scaffold, but having the same target specificity as 1D3 might be used.

Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module is a variant of DARPin 1D3 which binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module comprises a variant of the amino acid sequence of SEQ ID No. 2 which binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module is or is derived from DARPin 1D3, and
wherein said trimerization domain is or is derived from the capsid protein SHP of lambdoid phage 21.

In other embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module comprises the amino acid sequence of SEQ ID No. 2, and
wherein said trimerization domain is or is derived from the capsid protein SHP of lambdoid phage 21.

The first and the second module of the recombinant proteins of the present disclosure are linked via a flexible linker. Principally any flexible linker can be used within the spirit of the present disclosure. Certain preferred flexible linkers are glycine-serine linkers. A particular preferred flexible linker is a (Gly₄Ser)₄-linker.

Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein said first module and said second module are separated by a flexible linker.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein said first module and said second module are separated by a glycine-serine linker.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein said first module and said second module are separated by a (Gly₄Ser)₄-linker.

The designed ankyrin repeat domain and the trimerization domain of the first module of the recombinant proteins of the present disclosure are separated by a short linker. Preferred short linkers of the present disclosure are linkers which are no longer than four, preferably no longer than three, and more preferably no longer than two or only one amino acid long. A particular preferred short linker is glycine. Another particularly preferred short linker is glycine-alanine.

Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein the designed ankyrin repeat domain and the trimerization domain of said first module are separated by a short linker.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein the designed ankyrin repeat domain and the trimerization domain of said first module are separated by a short linker selected from glycine and glycine-alanine.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein the designed ankyrin repeat domain and the trimerization domain of said first module are separated by a short linker consisting of glycine.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein the designed ankyrin repeat domain and the trimerization domain of said first module are separated by a short linker consisting of glycine-alanine.

The recombinant proteins of the present disclosure may be assembled in various orientations. In certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein said second module in N-terminal of said first module.

In other embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus,
wherein said trimerization domain is capable of forming stable trimers, and
wherein said second module in C-terminal of said first module.

The recombinant proteins of the present disclosure may also be composed as recited in the following embodiments. In certain embodiments the present disclosure relates to recombinant proteins comprising from the N- to the C-terminus
a) a designed ankyrin repeat domain which binds to the knob of an adenovirus,
b) trimerization domain,
c) optionally a second designed ankyrin repeat domain, and
d) a protein scaffold, a bioactive peptide or a small organic molecule.

In other embodiments the present disclosure relates to recombinant proteins comprising from the N- to the C-terminus
a) a designed ankyrin repeat domain which binds to the knob of an adenovirus,
b) trimerization domain,
c) a flexible linker,
d) optionally a second designed ankyrin repeat domain, and
e) a protein scaffold, a bioactive peptide or a small organic molecule.

In other embodiments the present disclosure relates to recombinant proteins comprising from the N- to the C-terminus
a) a designed ankyrin repeat domain which binds to the knob of an adenovirus,
b) a short linker,
c) trimerization domain,
d) a flexible linker,
e) optionally a second designed ankyrin repeat domain, and
f) a protein scaffold, a bioactive peptide or a small organic molecule.

In other embodiments the present disclosure relates to recombinant proteins comprising from the N- to the C-terminus
a) a designed ankyrin repeat domain which binds to the knob of an adenovirus,
b) a short linker,
c) trimerization domain,
d) a flexible linker,
e) a second designed ankyrin repeat domain, and
f) a protein scaffold, a bioactive peptide or a small organic molecule.

In other embodiments the present disclosure relates to recombinant proteins comprising from the N- to the C-terminus
a) a designed ankyrin repeat domain which binds to the knob of an adenovirus,
b) a short linker,
c) trimerization domain,
d) a flexible linker, and
e) a protein scaffold, a bioactive peptide or a small organic molecule.

In certain embodiments the present disclosure relates to recombinant proteins comprising from the N- to the C-terminus
a) a protein scaffold, a bioactive peptide or a small organic molecule,
b) optionally a second designed ankyrin repeat domain,
c) a designed ankyrin repeat domain which binds to the knob of an adenovirus, and
d) trimerization domain.

In certain embodiments the present disclosure relates to recombinant proteins comprising from the N- to the C-terminus
a) a protein scaffold, a bioactive peptide or a small organic molecule,
b) optionally a second designed ankyrin repeat domain,
c) a flexible linker,
d) a designed ankyrin repeat domain which binds to the knob of an adenovirus, and
e) trimerization domain.

In certain embodiments the present disclosure relates to recombinant proteins comprising from the N- to the C-terminus
a) a protein scaffold, a bioactive peptide or a small organic molecule,
b) optionally a second designed ankyrin repeat domain,
c) a flexible linker,
d) a designed ankyrin repeat domain which binds to the knob of an adenovirus,
e) a short linker, and
f) trimerization domain.

In certain embodiments the present disclosure relates to recombinant proteins comprising from the N- to the C-terminus
a) a protein scaffold, a bioactive peptide or a small organic molecule,
b) a second designed ankyrin repeat domain,
c) a flexible linker,
d) a designed ankyrin repeat domain which binds to the knob of an adenovirus,
e) a short linker, and
f) trimerization domain.

In certain embodiments the present disclosure relates to recombinant proteins comprising from the N- to the C-terminus
a) a protein scaffold, a bioactive peptide or a small organic molecule,
b) a flexible linker,
c) a designed ankyrin repeat domain which binds to the knob of an adenovirus,
d) a short linker, and
e) trimerization domain.

In certain embodiments the present disclosure relates to recombinant proteins comprising from the N- to the C-terminus
a) an antibody fragment,
b) a flexible linker,
c) a designed ankyrin repeat domain which binds to the knob of an adenovirus,
d) a short linker, and
e) trimerization domain.

In certain embodiments said antibody of antibody fragment is an antibody fragment. In certain embodiments said antibody of antibody fragment is a scFv. Therefore in certain embodiments the present disclosure relates to recombinant proteins comprising from the N- to the C-terminus
a) a scFv antibody fragment,
b) a flexible linker,
c) a designed ankyrin repeat domain which binds to the knob of an adenovirus,
d) a short linker, and
e) trimerization domain.

In other embodiments the present disclosure relates to recombinant proteins comprising from the N- to the C-terminus
a) a scFv antibody fragment,
b) a flexible linker,
c) a designed ankyrin repeat domain which binds to the knob of an adenovirus,
d) a short linker, and
e) a trimerization domain which is or is derived from the capsid protein SHP of lambdoid phage 21.

In other embodiments the present disclosure relates to recombinant proteins comprising from the N- to the C-terminus
a) a scFv antibody fragment,
b) a flexible linker,
c) a designed ankyrin repeat domain which binds to the knob of an adenovirus,
d) a short linker, and
e) a trimerization domain comprising amino acid sequence of SEQ ID No. 1.

In certain embodiments the present disclosure relates to recombinant proteins comprising from the N- to the C-terminus
a) a scFv antibody fragment,
b) a flexible linker,
c) a designed ankyrin repeat domain which is or is derived from DARPin 1D3,
d) a short linker, and
e) trimerization domain.

In certain embodiments the present disclosure relates to recombinant proteins comprising from the N- to the C-terminus
a) a scFv antibody fragment,
b) a flexible linker,
c) a designed ankyrin repeat domain which comprises the amino acid sequence of SEQ ID No. 2,
d) a short linker, and
e) trimerization domain.

In certain embodiments the present disclosure relates to recombinant proteins comprising from the N- to the C-terminus
a) a scFv antibody fragment,
b) a flexible linker,
c) a designed ankyrin repeat domain which is a variant of DARPin 1D3 and which binds to the knob of an adenovirus,
d) a short linker, and
e) trimerization domain.

In certain embodiments the present disclosure relates to recombinant proteins comprising from the N- to the C-terminus
a) a scFv antibody fragment,
b) a flexible linker,
c) a designed ankyrin repeat domain which is or is derived from DARPin 1D3,
d) a short linker, and
e) a trimerization domain which is or is derived from the capsid protein SHP of lambdoid phage 21.

In certain embodiments the present disclosure relates to recombinant proteins comprising from the N- to the C-terminus
a) a scFv antibody fragment,
b) a flexible linker which is a glycine-serine linker,
c) a designed ankyrin repeat domain which is or is derived from DARPin 1D3,
d) a glycine or glycine-alanine linker, and
e) a trimerization domain which is or is derived from the capsid protein SHP of lambdoid phage 21.

In certain embodiments the present disclosure relates to recombinant proteins comprising from the N- to the C-terminus
a) a scFv antibody fragment,
b) a flexible linker which is a glycine-serine linker,
c) a designed ankyrin repeat domain which is or is derived from DARPin 1D3, and
d) a trimerization domain which is or is derived from the capsid protein SHP of lambdoid phage 21.

As already indicated the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

The recombinant proteins therefore optionally comprise a second designed ankyrin repeat domain.

If the second designed ankyrin repeat domain is present the present disclosure relates to recombinant proteins comprising
a) a first module comprising a designed ankyrin repeat domain and a trimerization domain,
b) a second module comprising
   i. a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

If the second designed ankyrin repeat domain is absent the present disclosure relates to recombinant proteins comprising
a) a first module comprising a designed ankyrin repeat domain and a trimerization domain, and
b) a second module comprising a protein scaffold, a bioactive peptide or a small organic molecule,
wherein said designed ankyrin repeat domain of said first module binds to the knob of an adenovirus, and
wherein said trimerization domain is capable of forming stable trimers.

One important component of the recombinant protein of the present disclosure are an antibody, an antibody fragment, a bioactive peptide or a small organic molecule. The prior art does not disclose such components in molecules that are similar to those described herein. The most similar compounds (see e.g. Proc. Natl. Acad. Sci. 110, E869-E877 (2013)) contains two designed ankyrin repeat domains. One of these designed ankyrin repeat domains binds to the knob of an adenovirus, whereas the other, second designed ankyrin repeat domains serves as retargeting domain with specificity for a different target protein.

This nature of the retargeting domain limits the use of respective adapter molecules. DARPins are able to specifically bind to certain target proteins. However, DARPins are less explored than other molecules, such as antibodies and antibody fragments. Antibody-based moieties can for example also bind to non-proteinaceous structures, such as sugars and lipids. Demonstrated herein is that bioactive peptides and small organic molecules can likewise be targeted by the constructs of the present disclosure. Using such entities open the door for many new uses. Non-proteinaceous moieties, such as organic molecules, can likewise be targeted by such retargeting moieties.

The antibody, the antibody fragment, the bioactive peptide or the small organic molecule can be attached, linked or fused to the recombinant proteins of the present invention by various means. As described herein, said antibody, antibody fragment, bioactive peptide or small organic molecule can either replace the designed ankyrin repeat domain in the second module, or can be linked, fused or attached the designed ankyrin repeat domain in the second module.

In cases where the antibody, the antibody fragment, the bioactive peptide or the small organic molecule replaces the designed ankyrin repeat domain in the second module, several technical hurdles need to be addressed. For example, when an antibody fragment, such as an scFv replaces the designed ankyrin repeat domain in the second module, this may lead to intra- and intermolecular misfolding and to aggregation. Creating a scFv by fusing VL and VH domains together with a linker causes the formation of dimers and multimers, where the VL domain of one chain binds to the VH domain of another chain. This is particular the case in the present context, where three scFv are displayed on the knob of the adenovirus in close proximity. This also leads to reduced stability. Also the direct fusion of functional domains in the absence of a linker may lead to many undesirable outcomes, including misfolding of the fusion proteins, low yield in protein production, or impaired bioactivity. See for example J Mol Biol (2001) 305, 989-1010.

The antibody, the antibody fragment and the bioactive peptide are typically peptides or polypeptides. If the second module comprises an antibody, an antibody fragment or a bioactive peptide, then said antibody, antibody fragment or bioactive peptide can be fused to the other part of the recombinant protein by classical genetic engineering technologies to form, after expression, one single protein.

However, it is also shown herein that retargeting moieties can also be linked to the recombinant proteins of the present disclosure by other means, such as by chemical conjugation. Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a bioactive peptide or a small organic molecule,
wherein said designed ankyrin repeat domain of said first module binds to the knob of an adenovirus, wherein said trimerization domain is capable of forming stable trimers, and
wherein said bioactive peptide or small organic molecule is linked to said second module via chemical conjugation.

Maleimide conjugation is one possibility, by which small organic molecules can be linked to polypeptides. Therefore, in certain embodiments the present disclosure relates to recombinant proteins comprising
a) a first module comprising a designed ankyrin repeat domain and a trimerization domain,
b) a second module comprising
   i. optionally a second designed ankyrin repeat domain, and
   ii. a bioactive peptide or a small organic molecule,
wherein said designed ankyrin repeat domain of said first module binds to the knob of an adenovirus, wherein said trimerization domain is capable of forming stable trimers, and
wherein said bioactive peptide or small organic molecule is linked to said second module via maleimide conjugation.

The second module of the recombinant proteins disclosed herein may comprise an antibody of antibody fragment. Said antibody fragment may be any antibody fragment known to the skilled person and include but are not limited to Fab fragments, F(ab)2 fragments, Fd fragments, single domain antibodies, maxibodies, minibodies, intrabodies, diabodies, scFv's, bis-scFv's and antibodies from camelids, such as nanobodies. Preferred antibody fragments of the present disclosure are scFv's. In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. an antibody,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody,
wherein said designed ankyrin repeat domain of said first module binds to the knob of an adenovirus, and
wherein said trimerization domain is capable of forming stable trimers.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. an antibody fragment,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody fragment, wherein said designed ankyrin repeat domain of said first module binds to the knob of an adenovirus, and
wherein said trimerization domain is capable of forming stable trimers.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. an antibody fragment, wherein said antibody fragment is a scFv,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus, and
wherein said trimerization domain is or is derived from the capsid protein SHP of lambdoid phage 21.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus, and
wherein said trimerization domain comprises the amino acid sequence of SEQ ID No. 1.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the an adenovirus comprising the knob of an adenovirus of serotype 5, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5, and
wherein said trimerization domain is or is derived from the capsid protein SHP of lambdoid phage 21.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said designed ankyrin repeat domain of said first module is or is derived from DARPin 1D3.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said designed ankyrin repeat domain of said first module comprises the amino acid sequence of SEQ ID No. 2.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5,
wherein said trimerization domain is capable of forming stable trimers, and
wherein said designed ankyrin repeat domain of said first module is or is derived from DARPin 1D3.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5,
wherein said trimerization domain is capable of forming stable trimers,
wherein said designed ankyrin repeat domain of said first module is or is derived from DARPin 1D3, and
wherein said trimerization domain is or is derived from the capsid protein SHP of lambdoid phage 21.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said first module and said second module are separated by a (Gly₄Ser)₄-linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers,
wherein said trimerization domain is or is derived from the capsid protein SHP of lambdoid phage 21, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers,
wherein said trimerization domain is or is derived from the capsid protein SHP of lambdoid phage 21, and
wherein said first module and said second module are separated by a (Gly₄Ser)₄-linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a (Gly₄Ser)₄-linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein the designed ankyrin repeat domain and the trimerization domain of said first module are separated by a short linker,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein the designed ankyrin repeat domain and the trimerization domain of said first module are separated by a short linker,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a (Gly₄Ser)₄-linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein the designed ankyrin repeat domain and the trimerization domain of said first module are separated by a short linker selected from glycine and glycine-alanine,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein the designed ankyrin repeat domain and the trimerization domain of said first module are separated by a short linker selected from glycine and glycine-alanine,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a (Gly₄Ser)₄-linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5,
wherein the designed ankyrin repeat domain and the trimerization domain of said first module are separated by a short linker selected from glycine and glycine-alanine,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a glycine-serine linker.

In other embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus of serotype 5,
wherein the designed ankyrin repeat domain and the trimerization domain of said first module are separated by a short linker selected from glycine and glycine-alanine,
wherein said trimerization domain is capable of forming stable trimers,
wherein said designed ankyrin repeat domain of said first module is or is derived from DARPin 1D3,
wherein said trimerization domain is or is derived from the capsid protein SHP of lambdoid phage 21, and
wherein said first module and said second module are separated by a (Gly₄Ser)₄-linker.

In certain embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said scFv is specific for HER2.

In certain embodiments, the present disclosure relates to recombinant proteins comprising
a) a first module comprising a designed ankyrin repeat domain and a trimerization domain, and
b) a second module comprising an antibody fragment, wherein said antibody fragment is a scFv,
wherein said designed ankyrin repeat domain of said first module binds to the knob of an adenovirus,
wherein said trimerization domain is capable of forming stable trimers, and
wherein said scFv specific for HER2 comprises the binding domain of 4D5 LH.

The second module of the recombinant proteins disclosed herein may comprise a peptide. Preferably the peptide is a bioactive peptide. An exemplary bioactive peptide is neurotensin. Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a peptide,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a peptide,
wherein said designed ankyrin repeat domain of said first module binds to the knob of an adenovirus, and
wherein said trimerization domain is capable of forming stable trimers.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a bioactive peptide,
wherein said designed ankyrin repeat domain of said first module binds to the knob of an adenovirus, and,
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module binds to the knob of an adenovirus, and
wherein said trimerization domain is capable of forming stable trimers.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a bioactive peptide wherein said bioactive peptide is neurotensin,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide wherein said bioactive peptide is neurotensin,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide wherein said bioactive peptide is a bioactive fragment of neurotensin,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module binds to the knob of an adenovirus, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module binds to the knob of an adenovirus of serotype 5, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module is or is derived from DARPin 1D3, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module binds to the knob of an adenovirus of serotype 5,
wherein said first module and said second module are separated by a (Gly₄Ser)₄-linker, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein said designed ankyrin repeat domain of said first module is or is derived from DARPin 1D3,
wherein said first module and said second module are separated by a (Gly₄Ser)₄-linker, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a bioactive peptide,
wherein the designed ankyrin repeat domain and the trimerization domain of said first module are separated by a short linker,
wherein said designed ankyrin repeat domain of said first module is or is derived from DARPin 1D3,
wherein said first module and said second module are separated by a (Gly₄Ser)₄-linker, and
wherein said trimerization domain is or is derived from the capsid protein SHP of lambdoid phage 21 and is capable of forming stable trimers.

The second module of the recombinant proteins disclosed herein may comprise a small organic molecule. Said small organic molecule may be linked to said second module via chemical conjugation. Said small organic molecule may also be linked to said second module via maleimide conjugation. An exemplary small organic molecule is folic acid.

Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a small organic molecule,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said small organic molecule is linked to said second module via chemical conjugation,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said small organic molecule is linked to said second module via maleimide conjugation,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said small organic molecule is linked to said second module via chemical conjugation,
wherein said designed ankyrin repeat domain of said first module binds to the knob of an adenovirus, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said small organic molecule is linked to said second module via chemical conjugation,
wherein said designed ankyrin repeat domain of said first module binds to the knob of an adenovirus of serotype 5, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said small organic molecule is linked to said second module via chemical conjugation,
wherein said designed ankyrin repeat domain of said first module is or is derived from DARPin 1D3, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said first module and said second module are separated by a glycine-serine linker,
wherein said small organic molecule is linked to said second module via chemical conjugation,
wherein said designed ankyrin repeat domain of said first module binds to the knob of an adenovirus of serotype 5, and
wherein said trimerization domain is or is derived from the capsid protein SHP of lambdoid phage 21 and is capable of forming stable trimers.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a small organic molecule,
wherein said first module and said second module are separated by a (Gly₄Ser)₄-linker,
wherein said small organic molecule is linked to said second module via chemical conjugation,
wherein said designed ankyrin repeat domain of said first module binds to the knob of an adenovirus of serotype 5, and
wherein said trimerization domain is or is derived from the capsid protein SHP of lambdoid phage 21 and is capable of forming stable trimers.

In certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a small organic molecule,
wherein said designed ankyrin repeat domain of said first module binds to the knob of an adenovirus,
wherein said trimerization domain is capable of forming stable trimers, and
wherein said small organic molecule is folic acid.

The second module of the recombinant proteins disclosed herein may also comprise a protein scaffold. Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   i. a protein scaffold,
wherein said designed ankyrin repeat domain of said first module binds to the knob of an adenovirus, and
wherein said trimerization domain is capable of forming stable trimers,
with the proviso that the protein scaffold is not a designed ankyrin repeat domain.

In certain embodiments, the present disclosure relates to recombinant proteins comprising
a) a first module comprising a designed ankyrin repeat domain and a trimerization domain,
b) a second module comprising a protein scaffold,
wherein said designed ankyrin repeat domain of said first module binds to the knob of an adenovirus, and
wherein said trimerization domain is capable of forming stable trimers, with the proviso that the protein scaffold is not a designed ankyrin repeat domain.

In other embodiments, the present disclosure relates to recombinant proteins comprising
a) a first module comprising a designed ankyrin repeat domain and a trimerization domain,
b) a second module comprising
   i. a second designed ankyrin repeat domain, and
   ii. a protein scaffold,
wherein said designed ankyrin repeat domain of said first module binds to the knob of an adenovirus, and
wherein said trimerization domain is capable of forming stable trimers.

In other embodiments, the present disclosure relates to recombinant proteins comprising
a) a first module comprising a designed ankyrin repeat domain and a trimerization domain, and
b) a second module comprising a protein scaffold,
wherein said designed ankyrin repeat domain of said first module binds to the knob of an adenovirus, and
wherein said trimerization domain is capable of forming stable trimers,
with the proviso that the protein scaffold is not a designed ankyrin repeat domain.

The recombinant proteins of the present disclosure comprise an antibody, an antibody fragment, a bioactive peptide or a small organic molecule, which serve as retargeting moieties. These retargeting moieties can be selected and may have specificity for any target of choice. Certain preferred targets are tumor antigens. Other preferred targets are surface proteins. Yet other preferred targets are antigens presented by MHC complexes. All these targets may also be post-translationally modified.

Therefore, in certain embodiments, the present disclosure relates to recombinant proteins comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, wherein said trimerization domain is capable of forming stable trimers, and
wherein said antibody, antibody fragment, bioactive peptide or small organic molecule is specific for a tumor antigen.

The recombinant proteins of the present disclosure are encoded by nucleic acids. Vectors comprising these nucleic acids are used to transfect adenoviruses which express the recombinant proteins.

Therefore, in certain embodiments, the present disclosure relates to a nucleic acid encoding a recombinant protein of the present disclosure. The present disclosure also relates to a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, wherein said trimerization domain is capable of forming stable trimers, and
wherein said antibody, antibody fragment, bioactive peptide or small organic molecule is specific for a tumor antigen.

In other embodiments, the present disclosure relates to a vector comprising a nucleic acid encoding a recombinant protein of the present disclosure. The present disclosure also relates to a vector comprising a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, wherein said trimerization domain is capable of forming stable trimers, and
wherein said antibody, antibody fragment, bioactive peptide or small organic molecule is specific for a tumor antigen.

In another embodiments, the present disclosure relates to an adenovirus comprising a nucleic acid encoding a recombinant protein of the present disclosure. In yet another embodiments, the present disclosure relates to an adenovirus comprising a vector comprising a nucleic acid encoding a recombinant protein of the present disclosure. In certain embodiments said adenovirus carries a TAYT mutation.

In certain embodiments, the present disclosure also relates to an adenovirus vector comprising a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, wherein said trimerization domain is capable of forming stable trimers, and
wherein said antibody, antibody fragment, bioactive peptide or small organic molecule is specific for a tumor antigen.

In certain embodiments, the present disclosure also relates to an adenovirus vector comprising a vector comprising a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, wherein said trimerization domain is capable of forming stable trimers, and
wherein said antibody, antibody fragment, bioactive peptide or small organic molecule is specific for a tumor antigen.

In certain embodiments, the present disclosure also relates to an adenovirus vector comprising a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus or serotype 5, wherein said trimerization domain is capable of forming stable trimers, and
wherein said antibody, antibody fragment, bioactive peptide or small organic molecule is specific for a tumor antigen.

In certain embodiments, the present disclosure also relates to an adenovirus vector comprising a vector comprising a nucleic acid encoding
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus or serotype 5, wherein said trimerization domain is capable of forming stable trimers, and
wherein said antibody, antibody fragment, bioactive peptide or small organic molecule is specific for a tumor antigen.

The recombinant proteins of the present disclosure can be expressed in procaryotic cells, such as Escherichia coli, and in eukaryotic cells. Preferred eukaryotic cells are CHO cells. Other preferred eukaryotic cells are HEK293 cells, HEK293 T cells, HEK293 F cells, CHO-S cells and Sf9 cells. Therefore, in certain embodiments the present disclosure provides a eukaryotic cell expressing the recombinant protein of the present disclosure. In certain other the present disclosure provides a CHO cell expressing the recombinant protein of the present disclosure. In certain embodiments, the present disclosure relates to a eukaryotic cell expressing a recombinant protein comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

In certain embodiments, the present disclosure relates to a CHO cell expressing a recombinant protein comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers.

The recombinant proteins of the present disclosure, the nucleic acids encoding the recombinant proteins of the present disclosure, the vectors containing the nucleic acids of the present disclosure and the adenoviruses containing the recombinant proteins, the nucleic acids or the vectors of the present disclosure have numerous uses, such as the use in an adenoviral delivery system. Therefore, in certain embodiments the present disclosure provides the use of the recombinant proteins of the present disclosure in an adenoviral delivery system. In other embodiments the present disclosure provides the use of the nucleic acids encoding the recombinant proteins of present disclosure in an adenoviral delivery system. In other embodiments the present disclosure provides the use of the vectors containing the nucleic acids of the present disclosure in an adenoviral delivery system. In other embodiments the present disclosure provides the use of the adenoviruses containing the recombinant proteins, the nucleic acids or the vectors of the present disclosure in an adenoviral delivery system.

The recombinant proteins of the present disclosure, the nucleic acids encoding the recombinant proteins of the present disclosure, the vectors containing the nucleic acids of the present disclosure, the adenoviruses containing the recombinant proteins, the nucleic acids or the vectors of the present disclosure, and the eukaryotic cells containing the adenoviruses of the present disclosure also have a use in medicine, since the retargeting moiety can guide the cells expressing the recombinant proteins of the present disclosure to any desired target molecule, e.g. a target protein. The so directed adenoviral vectors can then exert their function, which is dependent on the specific molecules, at the desired site within the human body. Target proteins that are associated with a disease or disorder are preferred target molecules. Therefore, in certain embodiments the present disclosure provides the recombinant proteins of the present disclosure for use in medicine. In certain embodiments, the present disclosure provides the nucleic acids encoding the recombinant proteins of the present disclosure for use in medicine. In certain embodiments, the present disclosure provides the vectors containing the nucleic acids of the present disclosure for use in medicine. In certain embodiments, the present disclosure provides the adenoviruses containing the recombinant proteins, the nucleic acids or the vectors of the present disclosure for use in medicine. In certain embodiments, the present disclosure provides a eukaryotic cell containing an adenovirus containing the recombinant proteins, the nucleic acids or the vectors of the present disclosure for use in medicine. In certain embodiments, the present disclosure provides a CHO cell containing an adenovirus containing the recombinant proteins, the nucleic acids or the vectors of the present disclosure for use in medicine.

In certain embodiments the present disclosure provides a method to treat a patient, said method comprising administering to a patient in need thereof a recombinant proteins of the present disclosure. In certain embodiments the present disclosure provides a method to treat a patient, said method comprising administering to a patient in need thereof a nucleic acid encoding a recombinant protein of the present disclosure. In certain embodiments the present disclosure provides a method to treat a patient, said method comprising administering to a patient in need thereof a vector containing a nucleic acid of the present disclosure. In certain embodiments the present disclosure provides a method to treat a patient, said method comprising administering to a patient in need thereof an adenovirus containing a recombinant protein, a nucleic acid or a vector of the present disclosure. In certain embodiments the present disclosure provides a method to treat a patient, said method comprising administering to a patient in need thereof a eukaryotic cell containing an adenovirus containing the recombinant proteins, the nucleic acids or the vectors of the present disclosure. In certain embodiments the present disclosure provides a method to treat a patient, said method comprising administering to a patient in need thereof a CHO cell containing an adenovirus containing the recombinant proteins, the nucleic acids or the vectors of the present disclosure.

Principally, the recombinant proteins of the present disclosure, the nucleic acids encoding the recombinant proteins of the present disclosure, the vectors containing the nucleic acids of the present disclosure, the adenoviruses containing the recombinant proteins, the nucleic acids or the vectors of the present disclosure, and the eukaryotic cells containing the adenoviruses of the present disclosure can be used in the treatment or prevention of any disease or disorder.

The present disclosure relates to a recombinant protein comprising
a) a first module comprising a designed ankyrin repeat domain and a trimerization domain,
b) a second module comprising
   i. optionally a second designed ankyrin repeat domain, and
   ii. a protein scaffold, a bioactive peptide or a small organic molecule,
wherein said designed ankyrin repeat domain of said first module binds to the knob of an adenovirus, and
wherein said trimerization domain is capable of forming stable trimers,
with the proviso that the protein scaffold is not a designed ankyrin repeat domain. In certain embodiments said protein is a monomeric protein.

In certain embodiments said first module and said second module are separated by a flexible linker.

In certain embodiments said flexible linker is a glycine-serine linker.

In certain embodiments said flexible linker is a (Gly Ser)₄-linker.

In certain embodiments said second module in N-terminal of said first module.

In certain embodiments said recombinant protein comprises from the N- to the C-terminus
a) a designed ankyrin repeat domain which binds to the knob of an adenovirus,
b) a short linker,
c) trimerization domain,
d) a flexible linker,
e) optionally a second designed ankyrin repeat domain, and
f) a protein scaffold or a small organic molecule.

In certain embodiments said second module in C-terminal of said first module.

In certain embodiments said recombinant protein comprises from the N- to the C-terminus
a) a protein scaffold or a small organic molecule,
b) optionally a second designed ankyrin repeat domain,
c) a flexible linker,
d) a designed ankyrin repeat domain which binds to the knob of an adenovirus,
e) a short linker, and
f) trimerization domain.

In certain embodiments said recombinant protein comprises from the N- to the C-terminus
a) a protein scaffold.

b) a flexible linker, and
c) a designed ankyrin repeat domain which binds to the knob of an adenovirus,
d) a short linker,
e) trimerization domain.

In certain embodiments said adenovirus is adenovirus serotype 5.

In certain embodiments said designed ankyrin repeat domain of said first module binds to the knob of adenovirus serotype 5.

In certain embodiments said designed ankyrin repeat domain of said first module is or is derived from DARPin 1D3.

In certain embodiments said designed ankyrin repeat domain of said first module comprises the amino acid sequence of SEQ ID No. 2.

In certain embodiments said trimerization domain of said first module is or is derived from the capsid protein SHP of lambdoid phage 21.

In certain embodiments said trimerization domain of said first module comprises the amino acid sequence of SEQ ID No. 1.

In certain embodiments the designed ankyrin repeat domain and the trimerization domain of said first module are separated by a short linker.

In certain embodiments said short linker is glycine or glycine-alanine.

In certain embodiments said protein scaffold is an antibody fragment or a single chain T cell receptor.

In certain embodiments said antibody fragment is a scFv.

In certain embodiments said scFv is specific for HER2.

In certain embodiments wherein said scFv specific for HER2 comprises the binding domain of 4D5 LH.

In certain embodiments said protein scaffold is a single chain T cell receptor.

In certain embodiments said second module comprises a peptide.

In certain embodiments said peptide is a bioactive peptide.

In certain embodiments said bioactive peptide is neurotensin.

In certain embodiments said second module comprises a small organic molecule.

In certain embodiments said small organic molecule is folic acid.

In certain embodiments said second module comprises a bioactive peptide or a small organic molecule, and wherein said bioactive peptide or small organic molecule is linked to said second module via chemical conjugation.

In certain embodiments said chemical conjugation is maleimide conjugation.

In certain embodiments said second designed ankyrin repeat domain is present.

In certain embodiments said antibody, antibody fragment, protein scaffold, bioactive peptide or small organic molecule is specific for a tumor antigen, tumor antigens, a surface proteins or an antigens presented by an MHC complex.

In certain embodiments the present disclosure relates to a nucleic acid encoding a recombinant protein as described herein above.

In certain embodiments the present disclosure relates to a vector comprising said nucleic acid.

In certain embodiments the present disclosure relates to an adenovirus comprising a recombinant protein, a nucleic acid or a vector as described herein above.

In certain embodiments said adenovirus carries a TAYT mutation.

In certain embodiments the present disclosure relates to a eukaryotic cell expressing or producing a recombinant protein, a nucleic acid, a vector or an adenovirus as described herein above.

In certain embodiments said eucaryotic cell is a CHO cell, a HEK cell or an insect cell, such as a Sf9 cell.

In certain embodiments the present disclosure relates to the use of a recombinant protein, a nucleic acid, a vector or an adenovirus as described herein above in an adenoviral delivery system.

In certain embodiments the present disclosure relates to the use of a recombinant protein, a nucleic acid, a vector or an adenovirus as described herein above in medicine.

### EXAMPLES

### Example 1: Retargeting Moieties

To demonstrate the broad application of the trimeric adapter system of the present disclosure, various cell lines and retargeting moieties were used. As a model system for modular targeting, we investigated human epidermal growth factor receptor 2 (HER2), neurotensin receptor 1 (NTR1), and folate receptor α (FTR) as target receptors.

HER2 is a receptor tyrosine kinase and has long been described as an important therapeutic target for gastric and breast cancer (Cancer Metastasis Rev. 34, 157-164 (2015)). A single-chain antibody was used to target HER2.

NTSR is a G protein-coupled receptor, involved in multiple biological regulatory functions as well as multiple pathophysiological processes (Sci. Adv. 7, 1-16 (2021)). A bioactive peptide was used to target NTSR.

Finally, FTR is a GPI-anchored protein of major interest in the field of oncology due to its important role to one-carbon metabolism and its overexpression in various solid tumors (Nat. Rev. Clin. Oncol. 17, 349-359 (2020)). A small molecule was used to target FTR.

### Example 2: Virus and Cell Lines

The replication-deficient HAdV-C5 vector contains an E1/E3 deletion and 4 mutations in the HVR7 (I421G, T423N, E424S and L426Y) and was generated as previously described (Nat. Commun. 9, 450 (2018)). The vector encodes the infrared fluorescent protein (iRFP670) or the Luciferase (luc) gene in the E1 region under the control of the cytomegalovirus major immediate early promoter (CMV). CHO cells, stably expressing human NTR1, were produced using the CHO Flp-In cell system (Thermo Fisher Scientific, Waltham (MA), USA). Cells were cultured in Dulbecco's modified Eagle's medium (DMEM), (respectively RPMI for KB cells) supplemented with tetracycline-free fetal calf serum (FCS product number) with a final concentration of 10% (v/v). NTR1 expression was induced by addition of 1 μg/ml tetracycline to the medium. KB cells and SKBR3 cells were obtained from the American Type Culture Collection (ATCC; www.atcc.org) and cultures were grown according to ATCC recommendations. 24 h before transduction, KB cells were starved using folate-free RPMI and 0% FCS. All cell lines have been verified to be free of mycoplasma contaminations.

### Example 3: Plasmid Construction and Expression of Bacterially Produced Adapters

For introducing a single N-terminal cysteine for thiol-maleimide conjugation, the plasmids pQIq (J. Mol. Biol. 413:826-43 (2011), encoding the trimeric adapters with G3 or E2_5, were modified at the DARPin's 5′ end by introducing a gene fragment. The gene fragment encoding a TEV protease cleavable His6-tag followed by a GCG motif for site specific conjugation (H2N-MRGSHHHHHHENLYFQGCG-COOH, SEQ ID No. 8) was introduced via EcoRI and BamHI restriction sites. All single-cysteine-containing and non-cysteine containing trimeric adapters were expressed in E. coli XL1-Blue as described (Proc. Natl. Acad. Sci. 110, E869-E877 (2013)). After expression, the cultures were centrifuged (4000×g, 10 min, 4° C.) and washed by resuspension of the cell pellets in ice-cold PBS, pH 7.4. Cell pellets were then resuspended in 50 mM Tris-HCl pH 8.0, 400 mM NaCl (TBS400), supplemented with 3 mg/ml lysozyme, 100 μg/mL DNase I and lysed by sonication and French Press. The obtained lysates were centrifuged (21,000×g, 30 min, 4° C.) and the supernatants applied to Ni-NTA Superflow (Qiagen, Hilden, Germany) metal ion affinity columns (4 mL). All columns were washed with each 15 column volumes (CV) of 50 mM Tris-HCl pH 8.0, 20 mM imidazole supplemented with 400 mM NaCl, 1 M NaCl or 20 mM NaCl, respectively. Then the adapters were eluted in 5 CV PBS pH 7.4, 500 mM imidazole. The eluted proteins were then transferred into dialysis tubes with a molecular weight cutoff of 6000-8000 Da, supplemented with 100 μg/mL TEV protease produced in-house and dialyzed overnight at 4° C. in 50 mM Tris-HCl pH 8.0, 150 mM NaCl, 0.5 mM EDTA, 1 mM DTT. The next morning, the TEV protease-cleaved His-tags and non-cleaved adapters were removed by running the content of the dialysis tubes over Ni-NTA Superflow metal ion affinity columns (1 ml). The flow-through was collected and concentrated by ultrafiltration (Amicon Centrifugal Filter Units, Merck Millipore, Billerica (MA), USA). Protein concentrations were determined by UV-Vis spectroscopy and purity was confirmed by SDS-PAGE analysis.

### Example 4: Expression and Purification of Adapters Expressed in Mammalian Cell

The trimeric adapters were cloned into pcDNA3.1, using the scFv 4D5 sequence previously reported (Adv. Cancer Res. 115, 39-67 (2012)). The scFv adapter was encoded with an HSA leader peptide, an N-terminal 3C-cleavable His6- and FLAG-tag. The retargeting domain is flanked by a BamHI and an HindIII site for ready exchange of the domain. The scFv-containing adapters were expressed in CHO-S cells as described (Protein Expr. Purif. 92, 67-76 (2013). Following expression for seven days, adapters were purified from the filtered supernatant using a Protein-L affinity chromatography (GE Healthcare, GE Healthcare Buckinghamshire, UK) with subsequent 3C cleavage of the tags during dialysis against 20 mM Hepes at pH 7.4. As an additional purification step, an anion exchange chromatography using a MonoQ column (GE Healthcare Buckinghamshire, UK) was performed. The purified protein was shock-frozen in liquid nitrogen and stored at −80° C. until usage.

### Example 5: Maleimide Conjugation of a Small Molecule or Bioactive Peptide to the Cysteine-Containing Retargeting Adapters

For site-specific conjugation to the N-terminal cysteines of purified adapters, folic acid was first coupled to form an amide with the N-terminus of a spacer peptide (sequence H2N-ASPASPASPASPASPASPA-COOH/SEQ ID No. 9) and derivatized with a maleimide group at the C-terminus (MW of Folate-Peptide-Maleimide=2164.96 g/mol), all custom synthesized by WuxXi Apptech (Shanghai, China). The final construct has the following structure:

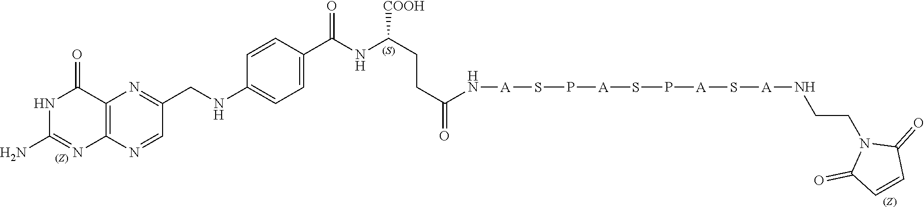

Protein samples were spiked with freshly dissolved DTT to a final concentration of 5 mM and reduced while shaking at 25° C. for 30 min. To remove DTT from the reduced samples, the buffer was exchanged to rigorously degassed PBS pH 7.4, 1 mM EDTA on a HiPrep 26/10 desalting column (GE Healthcare, GE Healthcare Buckinghamshire, UK) connected to an Äkta Explorer (GE Healthcare, GE Healthcare Buckinghamshire, UK) FPLC system. Desalted Protein samples (6-21 μM) were mixed with a 5-fold molar excess of maleimide-activated folate, maleimide-activated neurotensin over reduced cysteine. For conjugation, the reaction was incubated for 3 h at 25° C. with shaking. The conjugation mixtures were then quenched by the addition of a 5-fold molar excess of DTT over maleimide and incubated for 15 min at 25° C. with shaking. All steps were carried out under a nitrogen atmosphere. For purification of the conjugates from residual DTT, EDTA and quenched maleimide-activated FA and NT, the conjugation reactions were dialyzed in PBS pH 7.4 using dialysis tubes with a molecular weight (MW) cutoff of 7-14 kDa. During 24 h, the buffer was exchanged four times. The dialyzed conjugates were then concentrated to concentrations of 15-45 μM. Purity of conjugates was monitored by SDS-PAGE and the identity confirmed by ESI-MS.

### Example 6: ESI-MS Analysis

Protein masses were determined by time-of-flight (TOF) ESI-MS at the Functional Genomics Center Zurich (FGCZ). Prior to ESI-MS analysis, samples were desalted by C4 ZipTip (Merck Millipore, Billerica (MA), USA) reversed phase chromatography and eluted in MeOH: 2-propanol: 0.2% formic acid (30:20:50). The eluates were infused through a fused silica capillary (inner diameter 75 μm) at a flow rate of 1 μL/min and sprayed through a PicoTip with an inner diameter of 30 μm (New Objective, Littleton (MA), USA). Nano ESI-MS analysis of the samples was performed on a Synapt G2_Si mass spectrometer (Waters, Milford (MA), USA) and the data were recorded with MassLynx 4.2. Software (Waters, Milford (MA), USA). Mass spectra were acquired in positive-ion mode by scanning an m/z range from 100 to 5000 Da with a scan duration of 1 s and an interscan delay of 0.1 s. The spray voltage was set to 3 kV, the cone voltage to 40 V, and the source temperature to 80° C. The recorded m/z data were then deconvoluted into mass spectra by applying the maximum entropy algorithm MaxEnt1 (MaxLynx) with a resolution of the output mass of 0.5 Da/channel and Uniform Gaussian Damage Model at the half height of 0.7 Da.

### Example 7: Flow Cytometry Analysis of Expression

Cells were trypsinized for 20 min at 37° C. and washed with DMEM, centrifuged at 300 g for 3 min and resuspended in flow cytometry buffer (FC buffer) (PBS+1% BSA+0.05% azide) or FC buffer including surface staining reagent. Cells were incubated for 30 min at 4° C. in the dark, and then washed 3× with FC buffer before analyzed with the flow cytometer. HER2 was detected using a 1:200 dilution of FAB1129G (R&D systems), folate receptors with 1:50 dilution of FolateRSense™ 680 (Perkin Elmer, Waltham (MA), USA) and NTR1 was detected using fluorescently labeled neurotensin peptide HL488-NTS8-1335 in a 1:2 dilution.

### Example 8: Analysis of Viral Gene Delivery

Cells were seeded with 1.5×10⁴ cells per well of a 96-well plate, 24 h prior to infection. Adenoviral vectors encoding firefly luciferase or iRFP670 under the control of a CMV promoter were incubated with a retargeting adapter (retargeted), a non-binding adapter that only contained the knob-binding DARPin E2_5 (non-binding; J Mol Biol (2003) 332:489-503), resulting in blocking of the CAR-mediated uptake, or without any retargeting adapter (untargeted) for one hour at 4° C. The ratio of viral knob to adapters was 1:20 with a multiplicity of infection (MOI) of 2.5 plaque-forming units (PFU/cell). Viral particle-containing supernatants were removed 16 h post addition to the 96-well plate and replaced by fresh culture medium. Transgene expression was determined 72 h post transduction. Luciferase activity was determined by a luciferase assay (Promega, Fitchburg (WI), USA) according to the manufacturer's instructions. iRFP activity was measured by flow cytometry. Cells were washed with PBS and then detached using trypsin. Cells were then washed twice using PBS and then fixed using 2.5% PFA in PBS. After 20 min incubation at room temperature, cells were again washed with PBS containing 1% BSA and analyzed using the FACSCanto II 2L (BD).

### Example 9: Expression and Purification of Adapters Containing scFv's as Retargeting Module

In this experiment an adapter containing a single-chain variable fragment (scFv) as retargeting domain was cloned, expressed and purified. scFvs can readily be engineered from described antibodies. An scFv derived from the HER2-binding humanized mouse antibody hu4D5 was used. hu4D5 carries the binding domain of the therapeutic antibody trastuzumab (Endocr. Relat. Cancer 9, 75-85 (2002)).

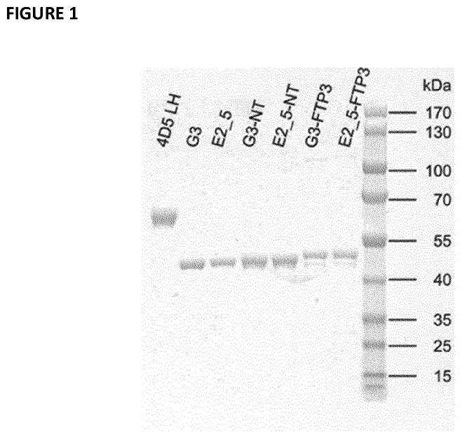

To exemplify the modular design, we used scFv 4D5 LH (LH meaning an orientation VH-linker-VL) also as an example for adapters expressed in eukaryotic cells. scFv 4D5 LH was cloned into the mammalian expression vector pcDNA3.1, and it was secreted using the HSA leader peptide. The leader peptide is followed by a His6-tag, a FLAG-tag, and a 3C protease cleavage site N-terminal to the retargeting module. This mammalian expression vector is designed to exchange the retargeting module with single-step cloning by flanking the retargeting module with BamHI and HindIII, allowing facile exchange of the retargeting module without interference with the knob-binding and trimerization modules. These plug-and-play designed mammalian expression vector allows fast and easy exchange of retargeting modules without the need for long optimization. Successful expression and purity of all constructs were determined by SDS-PAGE (FIG. 1).

### Example 10: Expression and Purification of Adapters Containing Bioactive Peptides or Small Molecules as Retargeting Module

In this experiment one adapter containing a bioactive peptide and another adapter containing a small molecule as retargeting domain was cloned, expressed and purified. To do so maleimide conjugation was used. A single cysteine was added to E2_5, a stable non-binding consensus DARPin functioning as a rigid spacer, and expressed the adapter containing this module in E. coli. For facile use, also adapters expressed in prokaryotes were generated, which carry an N-terminal His6-tag, and in which the retargeting module is flanked by BamHI and HindIII restriction sites. Since the adapters do not contain internal cysteine residues, the unique cysteine linked to the retargeting module allows defined coupling and expands retargeting possibilities to synthetic or toxic constructs.

Maleimide-coupled folate (FT-E2_5) was chosen as an example of a small molecule, and maleimide-activated neurotensin (NT-E2_5) as a peptide ligand, thereby exemplifying the broad applicability of the coupling strategy. Purity of the construct was determined by SDS-PAGE (FIG. 1). Correct coupling was confirmed by electrospray ionization mass spectrometry. See Table 1.

TABLE 1
Overview of the theoretical mass and electrospray
ionization mass spectrometry measured mass
of monomeric adapters after bioconjugation

| | Theoretical | Measured | Difference |
|---|---|---|---|
| Construct: | MW (Da) | MW (Da) | MW (Da) |
| G3-1D3nc-SHP1 | 30872 | | |
| Folate-G3-1D3nc-SHP1 | 44113 | 44113 | 0 |
| NT-GS-G3-1D3nc-SHP1 | 43953 | 43953 | 0 |
| E2_5-1D3nc-SHP1 | 42103 | | |
| Folate-E2_5-1D3nc-SHP1 | 44268 | 44269 | +1 |
| NT-GS-E2_5-1D3nc-SHP1 | 44107 | 44107 | 0 |

In conclusion, the generated vectors allow fast and easy adaptability of the trimeric construct for various research applications, e.g. screening of HAdV-C5 based gene delivery to a broad variety of different cells. Binding moieties may comprise of antibodies, antibody fragments, bioactive peptides or small molecules and can be attached or linked to the trimeric adapters by genetic fusion or chemical coupling. Additionally, these vectors circumvent complicated cloning procedures and can be expressed in various expression systems, including eucaryotic systems.

### Example 11: Transduction of Target Cells and Verification of Expression on the Cell Surface

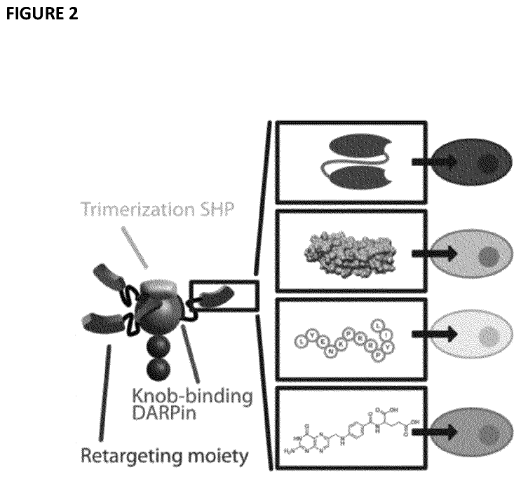

A schematic overview of the different types of constructs tested is shown in FIG. 2.

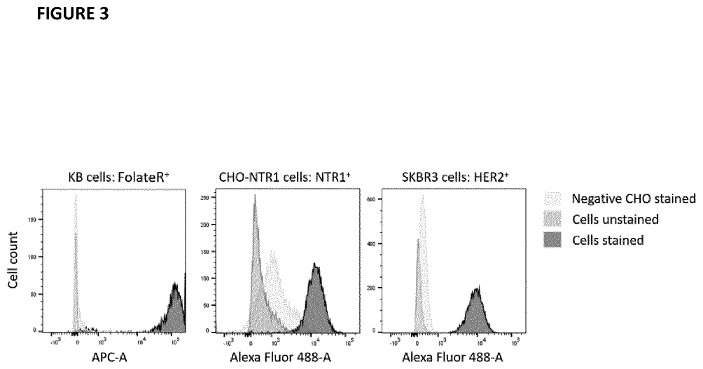

Expression of the constructs of the present invention on the cell surface of target cells was confirmed by flow cytometry (FIG. 3). Furthermore, the specific transduction capabilities of constructs comprising a scFv (4D5 LH), a peptide (NT-E2_5), and a small molecule (FTP3-E2_5) was analyzed.

Various controls were included in all transduction experiments. Unconjugated E2_5 adapters (non-binding E2_5) were used as a negative control (as it only blocks the binding site for CAR), HER2-binding DARPin G3 served as a HER2 positive control, and untargeted vectors which were not incubated with any adapters were also applied as controls for CAR-driven uptake. This latter comparison served as a reference to natural transduction efficiency of HAdV-C5.

Specificity of transduction can be maintained and transduction of potential free knob domains can be precluded using unconjugated consensus DARPin E2_5 as retargeting module. E2_5-fused adapters block the CAR-binding site, inhibiting a major host-virion interaction. A reduced transduction when using E2_5, compared to untargeted vector, shows effective knob binding and therefore successful manipulation of virion-host interactions.

The purified constructs were incubated with a first-generation ΔE1/E3 HAdV-C5 vector, encoding either infrared fluorescent protein 670 (iRFP) or luciferase, forming a stable adapter-vector complex. After incubating a 20-fold excess of adapter per HAdV-C5 fiber knob for 1 h at 4° C., the adapter-vector complexes were added to different cell populations using a multiplicity of infection (MOI) of 2.5 plaque-forming units (PFU) per cell. After 16 h incubation, the medium was exchanged and vectors remaining in the medium were washed away. Cells were then kept for another 24 h for sufficient expression of reporter until they were taken for transduction analysis. Adapter-vector complex was added either to CHO Flp-In parental cells, CHO Flp-In expressing neurotensin receptor 1 (NTR+), folate receptor α expressing KB cells, or HER2 expressing breast cancer cell line SKBR3. All constructs were investigated for unspecific vector uptake through unpredicted interactions. Since CHO Flp-In parental cells do not express our targeted receptors, transduction of CHO Flp-In parental cells would therefore have to be due to unspecific interactions.

None of the tested variants showed transduction of CHO Flp-In parental cells, demonstrating targeted HAdV-C5 transduction depends on specific binding (FIG. 3). Next, we analyzed inhibition of transduction using the control adapter E2_5 to confirm covered knob domains of HAdV-C5 and being a prerequisite of specific binding interactions for transduction. Indeed, using non-binding E2_5 adapters did block fiber knob-mediated transduction in KB and SKBR3 cells drastically, although residual transduction through independent uptake mechanisms remains possible (FIG. 2B). Since CHO cells are not human, they do not express CAR and therefore there is no interaction which could be blocked. Thus, no further reduction using non-binding E2_5 control compared to the untargeted vector transduction could be reported.

In contrast, transduction was observed where specific binding was expected. Not only did the scFv-containing adapter 4D5 LH permit transduction into SKBR3 cells, but also coupling of the small molecule folate to DARPin E2_5 (FT-E2_5) increased the transduction capability of HAdV-C5 into KB cells, which express folate receptor, thereby demonstrating efficient and specific transduction using antibody fragments or chemically synthesized small molecules.

Chemically coupled adapters also showed effectiveness when using a maleimide activated peptide. If NT was coupled to DARPin E2_5 (NT-E2_5) at its single cysteine, the vector-adapter complex transduced NTR1-expressing CHO Flp-In cells, resulting in 27% positive transduced cells. Notably, the same HAdV-C5 could not transduce the cells without a targeting adapter.

Taken together, the trimeric adapter system is a facile tool for rational engineering of targeted HAdV-C5 transduction, independent of expression host, genetic fusion or chemical coupling or size of the retargeting module.

### Example 12: Bispecific Targeting

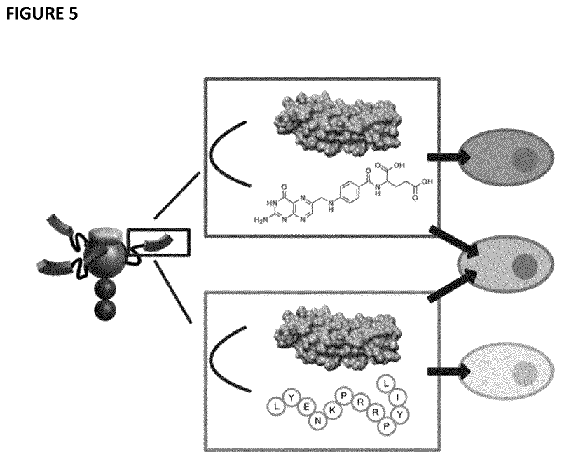

To prove a truly modular design and allow complex bivalent targeted transduction, we combined HER2-specific transduction mediated through the DARPin G3 and transduction via chemical coupling of folate or neurotensin. We replaced our rigid linker E2_5 with G3 while maintaining the unique cysteine. Using the same thiol-maleimide coupling, we generated a polyvalent adapter, binding HAdV-C5 knob, HER2, and either neurotensin receptor 1 by coupling neurotensin NT(8-13) (NT-G3), or folate receptor α by coupling folate (FTP3-G3) (FIG. 5).

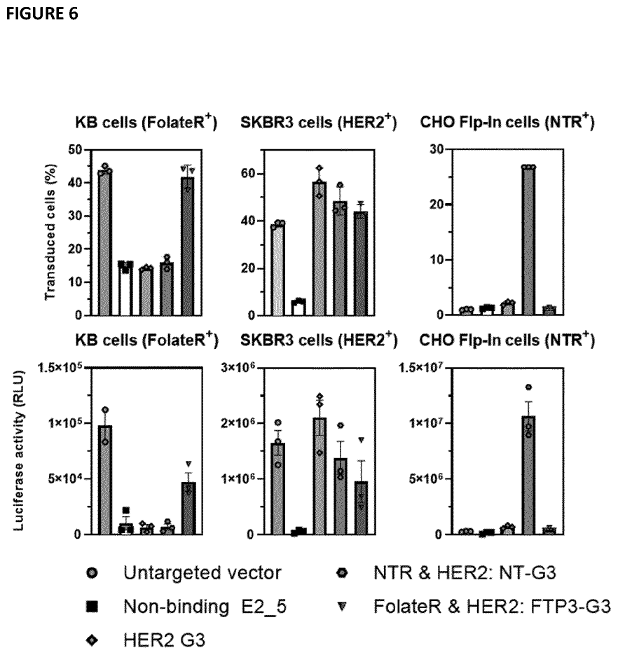

When the HER2-targeting module G3 as well as the chemically coupled module (NT or folate) are able to mediate specific transduction, we could verify the plug-and-play design by demonstrating successful and facile combination of different binding modules allowing ready design of polyvalent trimeric adapters. After complexation of NT-G3 or FT-G3 with HAdV-C5 encoding iRFP or luciferase, we compared transduction efficiencies on KB, SKBR3, and CHO Flp-In NTR+ (FIG. 6). To compare the transduction level, we also added adapter-vector complexes with unconjugated G3 or E2_5 (non-binding) and vector only (untargeted vector).

Target-specific transduction was achieved, since each construct should only interact with two of the three tested cell lines according to the expressed surface proteins, while unspecific transduction would therefore be observed on the third cell line. Using iRFP as a reporter, we could quantify the percentage of transduced cells, whereas luciferase activity allowed determination of reporter gene expression level with high sensitivity. Measuring both quantities thus allowed us to observe differences in the number of cells taking up the vector and amount of vector entering each cell. In line with our previous results, only on-target transduction was observed, and off-target transduction was inhibited by the blocked CAR interaction, independent of the cell type. Therefore specific and efficient transduction of all binding modules was maintained in this bispecific format.

### Example 13: Retargeting with a Replication-Competent Adeno Virus

In this experiment a replication competent adenovirus, HAdV-C5, was used to test the adapter molecules of the present disclosure. Tested were naked adenoviruses (no adapter), adenoviruses with a non-binding blocking adapter (DARPin E2_5, J Mol Biol (2003) 332:489-503), and adenoviruses with an EGFR retargeting adapter (Proc Natl Acad Sci USA (2013) 110: E869-77; please provide reference or sequence).

The EGFR retargeting adapter has the following sequence:

(SEQ ID No. 10)
DLGKKLLEAARAGQDDEVRILMANGADVNADDTWGWTPLHLAAYQGHLEI
VEVLLKNGADVNAYDYIGWTPLHLAADGHLEIVEVLLKNGADVNASDYIG
DTPLHLAAHNGHLEIVEVLLKHGADVNAQDKFGKTAFDISIDNGNEDLAE
ILQKLGGGGSGGGGSGGGGSGGGGSRSDLGKKLLEAARAGQDDEVRILMA
NGADANAYDHYGRTPLHMAAAVGHLEIVEVLLRNGADVNAVDTNGTTPLH
LAASLGHLEIVEVLLKYGADVNAKDATGITPLYLAAYWGHLEIVEVLLKH
GADVNAQDKFGKTPFDLAIDNGNEDIAEVLQGVRIFAGNDPAHTATGSSG
ISSPTPALTPLMLDEATGKLVVWDGQKAGSAVGILVLPLEGTETALTYYK
SGTFATEAIHWPESVDEHKKANAFAGSALSHAALP.

The ratio of viral knob to adapters was 1:100. The experiment was performed in the EGFR-positive RKO colon carcinoma cell line (ATCC, #CRL-2577). Cells were transduced either with the naked viruses or with the viruses plus the indicated adapters for 24h. Viruses contained a GLN reporter cassette (Cell Reports (2017) 19:1698-709).

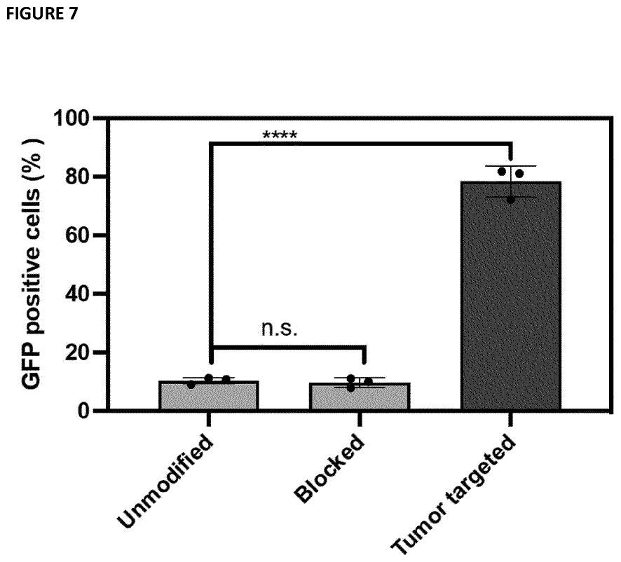

As transduction readout, GFP expression was measured over time in an Incucyte reader (Sartorius). Mock infected cells were included as a control. Results are shown in FIGS. 7 (GFP expression shown as % of GFP positive cells relative to confluency) and 8 (mean fluorescence). It can be seen that tumor targeting significantly increased transgene expression upon treatment of a replication-competent adenovirus with the adapter molecules of the present disclosure.

### Example 14: a Rigid Linker Facilitates Expression of Functional Adapter Molecules

In this experiment different linkers were compared Folate-G3-1D3nc-SHP1 (G3) and Folate-E2_5-1D3nc-SHP1 (E2_5) adapters were compared with Folate-1D3nc-SHP1 (A). Where Folate-1D3nc-SHP1 represents a direct replacement of the retargeting domain rather than using a 2NC repeat DARPin as a rigid linker. Control include cell background (C), untargeted virus (V) and virus covered with 1D3nc-SHP1 without any retargeting domain.

It can be seen that a second designed ankyrin repeat domain in the adapter molecules, which serves as a rigid linker, facilitates the functional expression the luciferase reporter moiety. Simple replacement of the retargeting domain with a small molecule is not sufficient for the most effective transduction of the target cell. The geometry of the DARPin serving as rigid spacer allows optimal interaction of the target receptor with the adenoviral vector, leading to effective uptake rather than just binding.

1: An adenovirus comprising a recombinant protein comprising
a) a first module comprising a designed ankyrin repeat domain and a trimerization domain,
b) a second module comprising a protein scaffold,
wherein said designed ankyrin repeat domain of said first module binds to the knob of an adenovirus, and
wherein said trimerization domain is capable of forming stable trimers,
with the proviso that the protein scaffold is not a designed ankyrin repeat domain.

2: An adenovirus according to claim 1, wherein said recombinant protein is displayed on the knob of said adenovirus.

3: An adenovirus according to claim 1, wherein said first module and said second module of the recombinant protein are separated by a flexible linker.

4: An adenovirus according to claim 1, wherein said second module of the recombinant protein is N-terminal of said first module.

5: An adenovirus according to claim 1, wherein said recombinant protein comprises from the N- to the C-terminus
a) a protein scaffold,
b) a flexible linker, and
c) a designed ankyrin repeat domain which binds to the knob of an adenovirus,
d) a short linker, and
e) trimerization domain.

6: An adenovirus according to claim 1, wherein said designed ankyrin repeat domain of said first module of the recombinant polypeptide binds to the knob of adenovirus serotype 5.

7: An adenovirus according to claim 1, wherein said trimerization domain of said first module of the recombinant polypeptide is or is derived from the capsid protein SHP of lambdoid phage 21.

8: An adenovirus according to claim 1, wherein said protein scaffold is an antibody fragment.

9: An adenovirus according to claim 8, wherein said antibody fragment is a scFv.

10: An adenovirus according to claim 9, wherein said scFv comprises the amino acid sequence of SEQ ID No: 4.

11: An eukaryotic cell comprising an adenovirus according to claim 1.

12: An adenoviral delivery system comprising an adenovirus according to claim 1.

13: An A method for treating a patient, said method comprising administering to a patient in need thereof an adenovirus according to claim 1.

14: A method for treating a patient, said method comprising administering to a patient in need thereof an eukaryotic cell according to claim 11.


SEQUENCE LISTING (10 sequences)

SEQ ID NO: 1 - protein, 103 aa, lambdoid phage 21
VRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 2 - protein, 157 aa, synthetic construct
RSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQG

SEQ ID NO: 3 - protein, 1255 aa, Homo sapiens [sequence omitted]

SEQ ID NO: 4 - protein, 106 aa, synthetic construct
DIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEL

SEQ ID NO: 5 - protein, 418 aa, Homo sapiens
MRLNSSAPGTPGTPAADPFQRAQAGLEEALLAPGFGNASGNASERVLAAPSSELDVNTDIYSKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLAVPMLFTMGEQNRSADGQHAGGLVCTPTIHTATVKVVIQVNTFMSFIFPMVVISVLNTIIANKLTVMVRQAAEQGQVCTVGGEHSTFSMAIEPGRVQALRHGVRVLRAVVIAFVVCWLPYHVRRLMFCYISDEQWTPFLYDFYHYFYMVTNALFYVSSTINPILYNLVSANFRHIFLATLACLCPVWRRRRKRPAFSRKADSVSSNHTLSSNATRETLY

SEQ ID NO: 6 - protein, 6 aa, Homo sapiens
RRPYIL

SEQ ID NO: 7 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 8 - protein, 19 aa, synthetic construct
MRGSHHHHHHENLYFQGCG

SEQ ID NO: 9 - protein, 19 aa, synthetic construct
ASPASPASPASPASPASPA

SEQ ID NO: 10 - protein, 435 aa, synthetic construct
DLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLGGGGSGGGGSGGGGSGGGGSRSDLGKKLLEAARAGQDDEVRILMANGADANAYDHYGRTPLHMAAAVGHLEIVEVLLRNGADVNAVDTNGTTPLHLAASLGHLEIVEVLLKYGADVNAKDATGITPLYLAAYWGHLEIVEVLLKHGADVNAQDKFGKTPFDLAIDNGNEDIAEVLQGVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP